(19) United States
(12) Patent Application Publication
(10) Pub. No.: US 2012/0252784 A1
(43) Pub. Date: Oct. 4, 2012

(54) 2,3,4-BENZOTHIADIAZEPINE-2,2-DIOXIDE DERIVATIVES

(76) Inventors: Jozsef FETTER, Goedoelloe (HU); Ferenc BERTHA, Budapest (HU); Balazs MOLNAR, Isaszeg (HU); Gyula SIMIG, Budapest (HU); Jozsef BARKOCZY, Budapest (HU); Balazs VOLK, Budapest (HU); Gyoergy LEVAY, Budakeszi (HU); Istvan GACSALYI, Budapest (HU); Gabor GIGLER, Budapest (HU); Hajnalka KOMPAGNE, Budapest (HU); Bernadett MARKO, Budapest (HU); Katalin NAGY, Ajka (HU); Peter KIRICSI, Budapest (HU); Laszlo Gabor HARSING, Budapest (HU); Gabor SZENASI, Ueroem (HU)

(21) Appl. No.: 13/418,869

(22) Filed: Mar. 13, 2012

Related U.S. Application Data
(63) Continuation-in-part of application No. PCT/HU2010/000103, filed on Sep. 29, 2010.

(30) Foreign Application Priority Data
Sep. 29, 2009 (HU) P0900616

(51) Int. Cl. A61K 31/554; C07D 513/04; C07C 309/81; C07C 303/02; C07C 309/24; A61P 25/24; A61P 25/28; A61P 25/16; A61P 25/18; A61P 25/00; A61P 25/22; C07D 285/36; C07D 285/14

(52) U.S. Cl. 514/211.08; 540/545; 562/833; 562/830; 562/41; 548/126

(57) ABSTRACT

Novel 2,3,4-benzothiadiazepines-2,2-dioxides and processes for the preparation thereof are disclosed. The compounds influence the central nervous system and may be used to treat or prevent Alzheimer's disease, Parkinson's disease or Huntington's disease, senile dementia not of the Alzheimer's type, vascular dementia, post-stroke dementia, Korsakoff's syndrome, Down's syndrome, schizophrenia, panic disorder, post-traumatic stress disorder, anxiety or depression.

### CROSS REFERENCE TO RELATED APPLICATIONS

This application is a continuation-in-part of PCT/HU2010/000103 filed 29 Sep. 2010, the contents of which are expressly incorporated herein, and claiming the benefit of the priority of Hungarian Patent Application P0900616 filed 29 Sep. 2009.

### FIELD OF THE INVENTION

The invention relates to 2,3,4-benzothiadiazepine-2,2-dioxide derivatives and enantiomers of the Formula (I), processes for their preparation, medicaments containing said compounds and methods of using the 2,3,4-Benzothiadiazepine-2,2-dioxide derivatives of Formula (I) are effective in the treatment of disorders of the central nervous system, including the treatment of Alzherimer's disease, Huntington's disease, Parkinson's disease, schizophrenia, vascular dementia, post-stroke dementia, senile dementia not of the Alzheimer's type, Down's syndrome, Korsakoff syndrome. Furthermore the compound s of the Formula (I) have anxiolytic and anti-depressant activity, and are useful in the treatment of Generalized Anxiety Disorder, panic disorder, post-traumatic stress disorder, unipolar depression and bipolar depression. Further objects of the present invention include the use of the compounds of the Formula (I) in the manufacture of a medicament containing the same as active ingredient.

### BACKGROUND OF THE INVENTION

The compounds of the Formula (I) of the present invention are new. The most closely related compound known from the prior art is 1,3-dihydro-2,3,4-benzothiadiazepine-2,2-dioxide of Formula (B) (J. F. King, A. Hawson, B. L. Huston, L. J. Danks, J. Komery, Can. J. Chem., 1971, 49, 943).

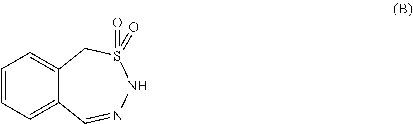

Substituted derivatives of the Formula (B) have never been described.

### OBJECTS OF THE INVENTION

An object of the present invention is to provide new 2,3,4-benzothiadiazepines of the Formula (I) effective in the treatment of disorders of the central nervous system.

A further object of the present invention is to provide a process for the preparation of the compounds of the Formula (I).

A further object of the invention is to provide pharmaceutical compositions comprising as active ingredient one or more compounds of the Formula (I) and/or enantiomers and/or pharmaceutically acceptable acid addition salts thereof in a therapeutically effective dose together with one or more pharmaceutically applicable diluents, excipients and/or inert carriers.

### SUMMARY OF THE INVENTION

The above mentioned objects can be achieved by the new compounds of the Formula (I),

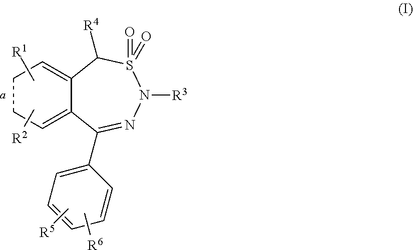

wherein,
‘a’ is a single or double bond,
R¹ and R² are each, independently, a hydrogen atom, halogen atom, cyano group, C₁₋₆ alkyl group, C₁₋₆ alkoxy group, trifluoromethyl group or a C₁₋₆ alkyl group substituted by one or more halogen atoms, or if
‘a’ is a single bond,
R¹ and R² together form a group of the formula ═N—S—N═ attached to the atoms of the bond ‘a’ according to the partial Formula (A)

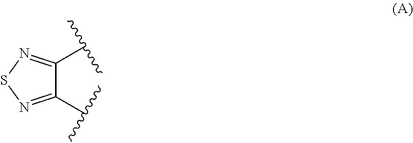

R³ and R⁴ are each, independently, a hydrogen atom, C₁₋₆ alkyl group, C₂₋₆ alkenyl group, C₂₋₆ alkynyl group, C₃₋₆ cycloalkyl group, aryl group or an aralkyl group where the alkyl is C₁₋₆ alkyl;
R⁵ and R⁶ are each, independently, a hydrogen atom, halogen atom, cyano group, C₁₋₆ alkyl group, C₁₋₆ alkoxy group, trifluoromethyl group or a C₁₋₆ alkyl group substituted by one or more halogen atom and/or enantiomers and/or pharmaceutically suitable acid addition salts thereof.

The present invention further relates to the compounds of the Formula (I) for use as a medicament.

The present invention further relates to the use of the compounds of the Formula (I) in the treatment and/or prevention of the disorder of the central nervous system.

Furthermore our invention relates to a method of treatment, which comprises administering to a patient suffering from a central nervous system disorder, a therapeutically effective dose of one or more compounds of Formula (I) and/or enantiomers and/or pharmaceutically acceptable acid additional salts thereof.

### DETAILED DESCRIPTION OF THE INVENTION

The present invention relates to compounds of the general Formula (I)

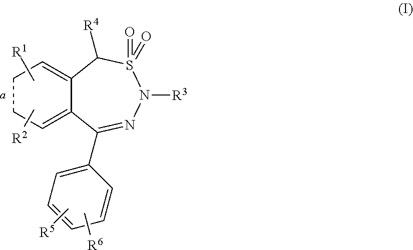

wherein
‘a’ is a single or double bond,
R¹ and R² are each, independently, a hydrogen atom, halogen atom, cyano group, C₁₋₆ alkyl group, C₁₋₆ alkoxy group, trifluoromethyl group, or other C₁₋₆ alkyl group substituted by one or more halogen atom or if
is a single bond,
R¹ and R² together form a group of the formula ═N—S—N═ attached to the atoms of the bond ‘a’ according to the partial Formula

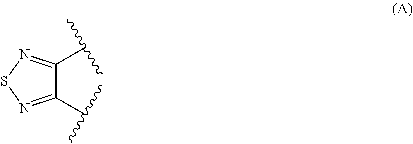

R³ and R⁴ are each, independently, a hydrogen atom, C₁₋₆ alkyl group, C₂₋₆ alkenyl group, C₂₋₆ alkynyl group, C₃₋₆-cycloalkyl group, aryl group or aralkyl group, R⁵ and R⁶ are each, independently, a hydrogen atom, halogen atom, cyano group, C₁₋₆ alkyl group, C₁₋₆ alkoxy group, trifluoromethyl group or a C₁₋₆ alkyl group substituted by one or more halogen atom and/or enantiomers and/or pharmaceutically acceptable acid addition salts thereof.

The definitions of the substituents referred to in the present specification are the following.

The expression “alkyl group” is a straight or branched saturated hydrocarbon chain having 1-6 carbon atoms, preferably 1-4 carbon atoms, e.g. methyl, ethyl, n-propyl, i-propyl, -butyl, 1-butyl, s-butyl, t-butyl group.
“Alkoxy group” is a hydroxy group substituted with an above defined alkyl group.
“Alkenyl group” represents a straight or branched-chain hydrocarbon group containing one double bond and having 2-6 carbon atoms, preferably 2-4 carbon atoms.
“Alkynyl group” represents a straight or branched-chain hydrocarbon group containing one triple bond and having 2 to 6 carbon atoms, preferably 2-4 carbon atoms.
“Aralkyl group” represents an alkyl group substituted at any position by an aromatic substituent, e.g. an optionally substituted phenyl group. Preferably the aralkyl group is an optionally substituted benzyl group where the substituents include at least one halogen atom, C₁₋₆ alkyl group, C₁₋₆ alkoxy group, hydroxy group, or a C₁₋₆ alkyl group substituted by one or more halogen atoms.
“Halogen atom” is a fluorine, chlorine, bromine or iodine atom, preferably chlorine or fluorine.
“Pharmaceutically acceptable” acid addition salts are salts formed with a non-toxic inorganic acid, e.g. hydrochloric acid, sulfuric acid, phosphoric acid, or with a non-toxic organic acid, e.g. acetic acid, fumaric acid, maleic acid, lactic acid, maleic acid, tartaric acid.
Preferable specific compounds of the Formula (I) are:
8-cyano-5-(4-fluoro-phenyl)-1,3-dihydro-2,3,4-benzothiadiazepine-2,2-dioxide;
7-chloro-5-(3-fluoro-phenyl)-1,3-dihydro-2,3,4-benzothiadiazepine-2,2-dioxide;
9-(4-methyl-phenyl)-5H,7H-[1,2,5]tluadiazolo[3,4-h]-2,3,4-benzothiadiazepine-6,6-dioxide;
9-(4-methoxy-phenyl)-5H,7H-[1,2,5]thiadiazolo[3,4-h][2,3,4]benzothiadiazepine-6,6-dioxide;
9-(3,4-dimethoxy-phenyl)-5H,7H-[1,2,5]thiadiazolo[3,4-h][2,3,4]benzothiadiazepine-6,6-dioxide;
9-(4-fluoro-phenyl)-5H,7H-[1,2,5]thiadiazolo[3,4-h][2,3,4]benzothiadiazepine-6,6-dioxide;
7-chloro-5-(4-fluoro-phenyl)-1,3-dihydro-2,3,4-benzothiadiazepine-2,2-dioxide;
7-fluoro-5-(4-fluoro-phenyl)-1,3-dihydro-2,3,4-benzothiadiazepine-2,2-dioxide;
7,8-dichloro-5-(4-fluoro-phenyl)-1,3-dihydro-2,3,4-benzothiadiazepine-2,2-dioxide;
7-chloro-5-(4-fluoro-phenyl)-3-methyl-1,3-dihydro-2,3,4-benzothiadiazepine-2,2-dioxide;
7-chloro-3-ethyl-5-(4-fluoro-phenyl)-1,3-dihydro-2,3,4-benzothiadiazepine-2,2-dioxide;
7-chloro-5-(4-fluoro-phenyl)-3-propyl-1,3-dihydro-2,3,4-benzothiadiazepine-2,2-dioxide;
7-chloro-5-(4-fluoro-phenyl)-3-(propynyl)-1,3-dihydro-2,3,4-benzothiadiazepine-2,2-dioxide;
7-chloro-5-(4-fluoro-phenyl)-3-isopropyl-1,3-dihydro-2,3,4-benzothiadiazepine-2,2-dioxide;
7-chloro-5-(4-fluoro-phenyl)-3-(3-methyl-but-2-enyl)-1,3-dihydro-2,3,4-benzothiadiazepine-2,2-dioxide;
7-chloro-5-(4-fluoro-phenyl)-3-(3-methyl-butyl)-1,3-dihydro-2,3,4-benzothiadiazepine-2,2-dioxide;
3-benzyl-7-chloro-5-(4-fluoro-phenyl)-1,3-dihydro-2,3,4-benzothiadiazepine-2,2-dioxide;
7-fluoro-5-(4-fluoro-phenyl)-3-methyl-1,3-dihydro-2,3,4-benzothiadiazepine-2,2-dioxide;
7-fluoro-3-ethyl-5-(4-fluoro-phenyl)-1,3-dihydro-2,3,4-benthiadiazepine-2,2-dioxide;
3-butyl-7-fluoro-5-(4-fluoro-phenyl)-1,3-dihydro-2,3,4-benzothiadiazepine-2,2-dioxide;
7-fluoro-5-(4-fluoro-phenyl)-3-(3-methyl-but-2-enyl)-1,3-dihydro-2,3,4-benzothiadiazepine-2,2-dioxide;
7-fluoro-5-(4-fluoro-phenyl)-3-(3-methyl-butyl)-1,3-dihydro-2,3,4-benzothiadiazepine-2,2-dioxide;
7-fluoro-5-(4-fluoro-phenyl)-3-hexyl-1,3-dihydro-2,3,4-benzothiadiazepine-2,2-dioxide;
3-benzyl-7-fluoro-5-(4-fluoro-phenyl)-1,3-dihydro-2,3,4-benzothiadiazepine-2,2-dioxide;
7-fluoro-5-(4-fluoro-phenyl)-3-(3-phenyl-propyl)-1,3-dihydro-2,3,4-benzothiadiazepine-2,2-dioxide;
5-(3-fluoro-phenyl)-7-chloro-3-methyl-1,3-dihydro-2,3,4-benzothiadiazepine-2,2-dioxide;
8-cyano-5-(4-fluoro-phenyl)-3-methyl-1,3-dihydro-2,3,4-benzothiadiazepine-2,2-dioxide;
8-fluoro-5-(4-fluoro-phenyl)-3-methyl-1,3-dihydro-2,3,4-benzothiadiazepine-2,2-dioxide;
5-(4-fluoro-phenyl)-8-chloro-3-methyl-1,3-dihydro-2,3,4-benzothiadiazepine-2,2-dioxide;
5-(4-fluoro-phenyl)-3-methyl-8-trifluoromethyl-1,3-dihydro-2,3,4-benzothiadiazepine-2,2-dioxide;
7,8-dichloro-5-(4-fluoro-phenyl)-3-methyl-1,3-dihydro-2,3,4-benzothiadiazepine-2,2-dioxide;
9-(4-fluoro-phenyl)-7-methyl-5H,7H-[1,2,5]thiadiazolo[3,4-h][2,3,4]benzothiadiazepine-6,6-dioxide;
7,8-dichloro-3-ethyl-5-(4-fluoro-phenyl)-1,3-dihydro-2,3,4-benzothiadiazepine-2,2-dioxide;
7-chloro-5-(4-fluoro-phenyl)-1,3-dimethyl-1,3-dihydro-2,3,4-benzothiadiazepine-2,2-dioxide;
7-chloro-1-ethyl-5-(4-fluoro-phenyl)-3-methyl-1,3-dihydro-2,3,4-benzothiadiazepine-2,2-dioxide
   and/or enantiomers and/or pharmaceutically acceptable acid addition salts thereof.
   The invention further relates to enantiomers of the compounds of the Formula (I) and the mixtures thereof.

### BRIEF DESCRIPTION OF THE DRAWINGS

FIG. 1 is a schematic diagram showing the reaction scheme for each step of the process for preparing the new compounds of the Formula (I) starting from the compounds of the Formula (IV).

FIG. 2 is a set of two bar graphs which compare the amount of taurine present in the frontal cortex of rats who were administered the compound of the Formula (I) according to Example 16 versus the amount of taurine present in the frontal cortex of rats who were not treated, that is an untreated control.

A further feature of the present invention is a process for the preparation of the compounds of the Formula (I), which consist of simple reaction steps providing high yield.

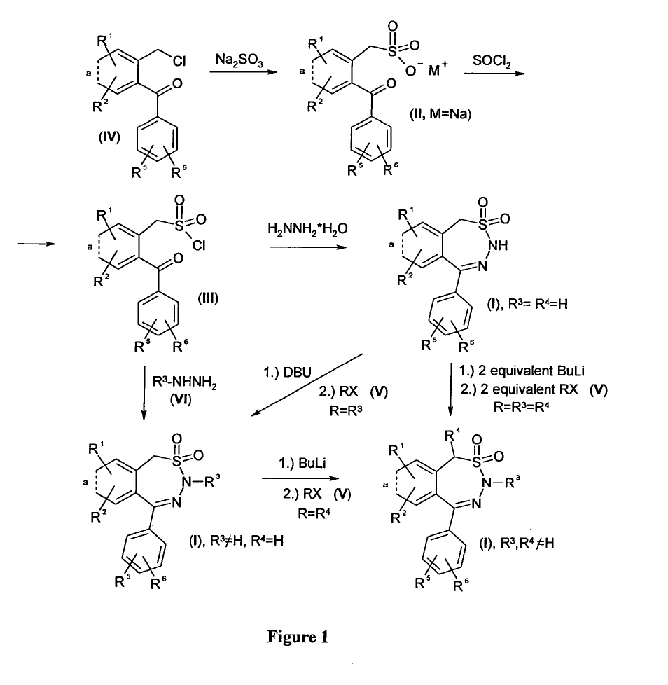

The reaction method used in the preparation of the compounds of the present invention is demonstrated in Reaction Scheme 1 as shown below and in FIG. 1:

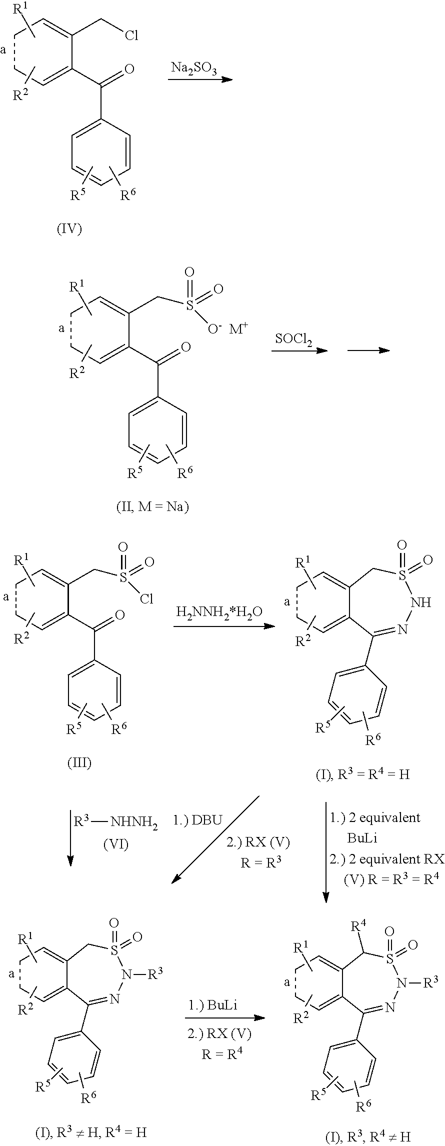

The compounds of the Formula (I) of the present invention, wherein R³ and R⁴ are hydrogen atoms and R¹, R², R⁵ and R⁶ are as defined above and pharmaceutically acceptable acid addition salts thereof are prepared by reacting the compound of the Formula (II), wherein R¹, R², R⁵ and R⁶ are as defined above and M represents monovalent or multivalent metals, preferably alkali metals, more preferably a sodium atom, with thionyl chloride, and subsequently the obtained compound of the Formula (III), wherein R¹, R², R⁵ and R⁶ are as defined above, is reacted with hydrazine.

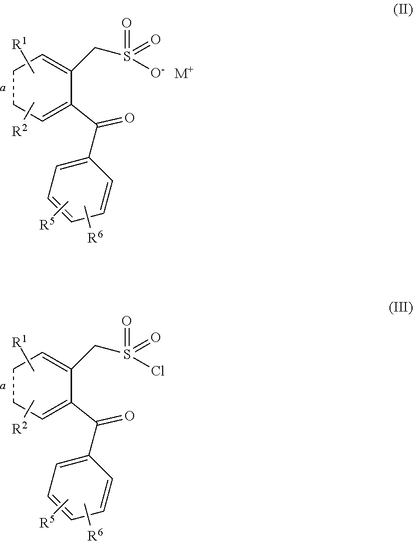

The compounds of the Formula (I) of the present invention, wherein R⁴ is hydrogen and R¹, R², R⁵, R⁶ and R³ are as defined above provided that R³ is different from hydrogen (I, R³≠H, R⁴=H) and pharmaceutically suitable acid addition salts thereof are prepared by reacting the 2,3,4-benzothiadiazepine-dioxide derivatives of the Formula (I), wherein R³ and R⁴ are hydrogen, R¹, R², R⁵ and R⁶ are as defined above (I, R³=R⁴=H), with agents suitable for introducing an alkyl group, alkenyl group, alkynyl group or aralkyl group, in the presence of a base.

Furthermore, the compounds of the Formula (I) of the present invention, wherein R⁴ is hydrogen and R¹, R², R⁵, R⁶ and R³ are as defined above provided that R³ is different from hydrogen (I, R³≠H, R⁴=H) and pharmaceutically suitable acid addition salts thereof can be prepared by reacting compounds of the formula (III), wherein R¹, R², R⁵ and R⁶ are as defined above, with a substituted hydrazine of the formula (VI), wherein R³ is as defined above.

R³—NH—NH₂ (VI)

The compounds of the Formula (I) of the present invention, wherein R³ and R⁴ are as defined above provided that R³ and R⁴ are identical and different from hydrogen and R¹, R², R⁵ and R⁶ are as defined above (I, R³=R≠H) and pharmaceutically suitable acid addition salts thereof are prepared by reacting the 2,3,4-benzothiadiazepine-dioxide derivatives of the Formula (I), wherein R³ and R⁴ are hydrogen, R¹, R², R⁵ and R⁶ are defined in claim 1 (I, R³=R⁴=H), with agents suitable for introducing an alkyl group, alkenyl group, alkynyl group or aralkyl group, in the presence of a base.

Furthermore, the compounds of the Formula (I) of the present invention, wherein R³ and R⁴ are as defined above provided that R³ and R⁴ are different from hydrogen and R¹, R², R⁵ and R⁶ are as defined above (I, R³≠H, R⁴≠H) and pharmaceutically suitable acid addition salts thereof can be prepared by reacting the 2,3,4-benzothiadiazepine-dioxide derivatives of the Formula (I), wherein R⁴ is hydrogen, R¹, R², R⁵, R⁶ and R³ are as defined above provided that R³ is different from hydrogen (I, R³≠H, R⁴=H), with agents suitable for introducing an alkyl group, alkenyl group or aralkyl group, in the presence of a base.

A further feature of the present invention are the compounds of the Formula (III),

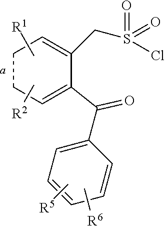

and the compounds of the Formula (II),

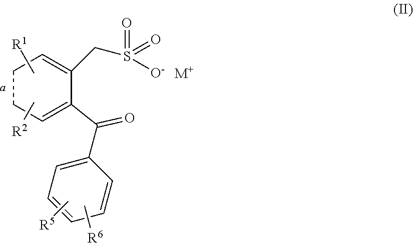

wherein M represents monovalent or multivalent metals, preferably alkali metals more preferably a sodium atom.
Preferred representatives of the compound of the Formula (II) are:
sodium [4-chloro-2-(4-fluoro-benzoyl)-phenyl]-methanesulfonate;
sodium [6-[(3,4-dimethoxy-phenyl)-carbonyl]-2,1,3-benzothiadiazole-5-yl]-methanesulfonate;
sodium [6-[(4-fluoro-phenyl)-carbonyl]-2,1,3-benzothiadiazole-5-yl]-methanesulfonate;
sodium [6-[(4-methoxy-phenyl)-carbonyl]-2,1,3-benzothiadiazole-5-yl]-methanesulfonate.

A further feature of the present invention is a process for the preparation of the compounds of the Formula (III), which comprises the step of reacting the compound of the Formula (II), wherein R¹, R², R⁵ and R⁶ are defined above with thionyl chloride.

A further feature of the present invention is a process for the preparation of the compounds of the Formula (II), wherein R¹, R², R⁵ and R⁶ are as defined above, which comprises the step of reacting the compound of Formula (IV), wherein R¹, R², R³ and R⁴ are as defined above with ammonium sulfite or alkali sulfite in the present of water or water-immiscible solvent, preferably in acetone, dioxane or in an aliphatic alcohol comprising 1 to 4 carbon atoms or in the mixture thereof.

The compounds of the Formula (IV)

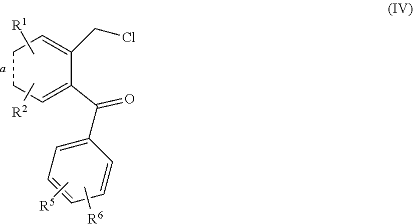

were prepared by a method known from the art for the synthesis of compounds with a similar structure (J Org. Chem., 1988, 53, 3621; J Org. Chem., 2003, 68, 4123). The [substituted 2-(substituted arylcarbonyl)-phenyl]methanesulfonate salts of the Formula (II) are prepared by the replacement of the halogen atom to sulfonic acid moiety in water, a water-miscible solvent (acetone, dioxane, alcohols) or in a mixture of water and a water-miscible solvent, by reacting the halogen compound with ammonium or alkali sulfites.

The compounds of Formula (IV) are novel excluding the following specific compounds which are known in the literature

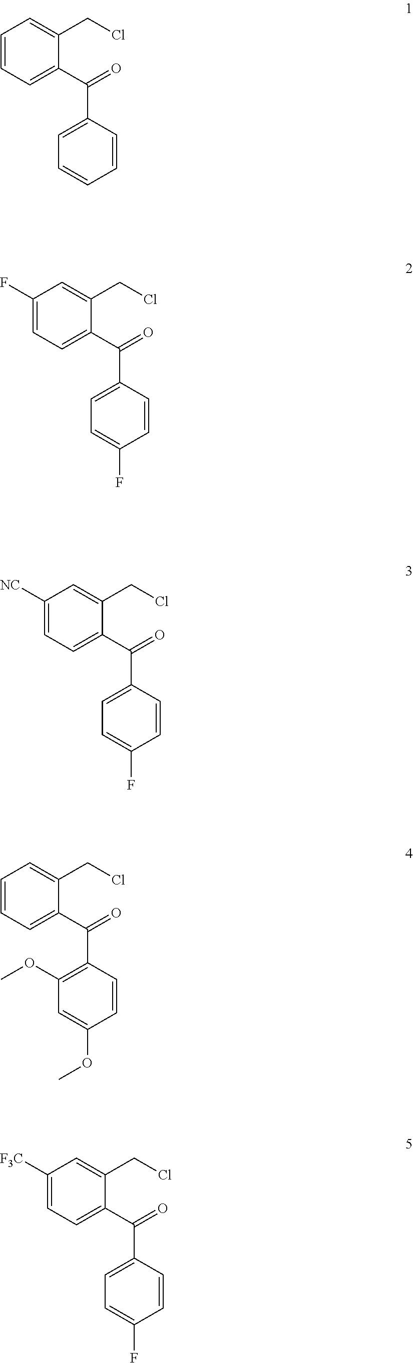

These specific compounds are disclosed in the following patents or articles:

### Compound IV-1:

Substituted [2-(aminomethyl)phenyl)phenylmethanones
H. Roehnert, F. Bahr, K. Unverferth, W. Kirsten,
Ger. (East) (1983), DD 158237 A1 19830105

### Compound IV-2:

### Toner for Electrostatic Image Development Using Aromatic Fluorine Compound as Charge-Controlling Agent

Minamitani, Toshiki; Yamaguchi, Kimitoshi; Kawase, Hiromitsu; Ogawara, Makoto; Ogawara, Makoto
Jpn. Kokai Tokkyo Koho (1991), JP 03007955 A 19910116

### Compound IV-3:

Process for the Preparation of 1-(3-dimethylaminopropyl)-1-(4-fluorophenyl)-1,3-dihydroisobenzofuran-5-carbonitrile (Citalopram)
Hilden, Leif; Huuhtanen, Tuomas; Rummakko, Petteri; Grumann, Arne; Pietikaeinen, Pekka
PCT Int. Appl. (2002), WO 2002060886 A1 20020808

### Compound IV-4:

Biosynthesis of Phenols. XX. Synthesis of Anthraquinones Through Carbanions of Ortho-Substituted Benzophenones
Davies, John Salmon; Davies, Vivian Harvey; Hassall, Cedric H.
Journal of the Chemical Society [Section] C: Organic (1969), (14), 1873-9

### Compound IV-5:

### Electrophotographic Developing Apparatus Having Aromatic Fluoride-Coated Toner-Supplying Member

Minamitani, Toshiki; Mochizuki, Chiharu
Jpn. Kokai Tokkyo Koho (1993), JP 05011499 A 19930122.

The methods of synthetising Formula (IV) are known for example according to the method disclosed in WO2002060886. In this reference there is an example on page 10 Example 2 second part how to synthetise 3-(chloromethyl)-4-[(4-fluorophenyl)carbonyl]benzonitile (3) starting from 2-(chloromethyl)-4-cyanobenzoyl chloride (6) and 4-fluoro phenylmagnesium bromide (7) in a Grignard reaction.

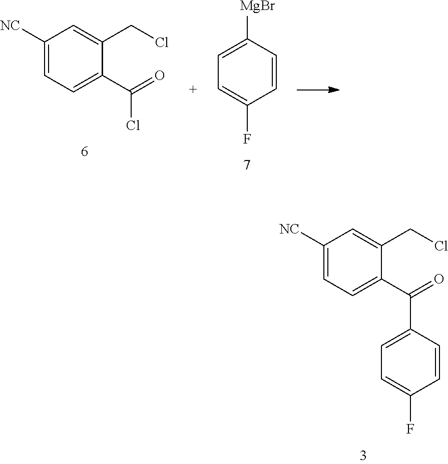

According to this method we synthetised the compounds of the Formula (IV). Now we add a concrete receipt for the synthesis of one of the compounds with Formula (IV), which is not known in the literature.

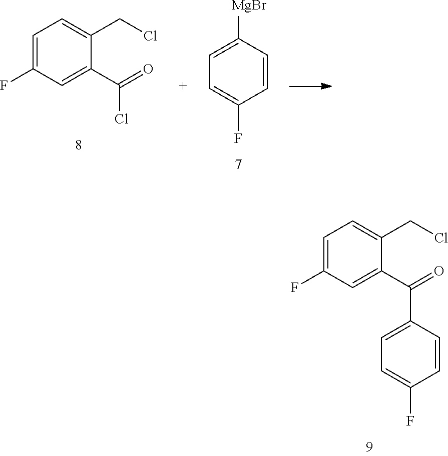

The compounds of Formula (II) are new and have not been described in the literature before. The metal salts of the general Formula (II) are formed by monovalent or multivalent metals, preferably alkali metals more preferably sodium atom.

The sulfonyl chlorides of the Formula (III) are prepared from the new benzylsulfonyl derivatives of the Formula (II) with thonyl chloride. The 2,3,4-benzothiadiazepine-2,2-dioxide derivatives of the Formula (I), wherein R³ and R⁴ represent hydrogen atom, are obtained from sulfonyl chlorides of the Formula (III) by closing the ring with hydrazine. Preferably the compounds of Formula (III) are not purified, but the crude product was reacted with hydrazine immediately.

An alternative one-pot method involves ring closure of sulfonyl chlorides of Formula (III) by reacting with compound of Formula (VI),

R³—NH—NH₂ (VI)

wherein R³ represents an alkyl, alkenyl, alkynyl or aralkyl group. This one-pot method yields 2,3,4-benzothiadiazepine-2,2-dioxide derivatives of the Formula (I), wherein R³ is an alkyl, alkenyl, alkynyl or aralkyl group and R⁴ represents hydrogen.

Optionally the 2,3,4-benzothiadiazepine-2,2-dioxide derivatives of the Formula (I), wherein R³ and R⁴ represent hydrogen, can be converted to compounds of the Formula (I), wherein R³ represents alkyl group, alkenyl group, alkynyl group or aralkyl group and R⁴ represents hydrogen atom, by reacting with an agent suitable for introducing an alkyl, alkenyl group or aralkyl group, in the presence of a base. In the reaction the agent suitable for introducing an alkyl group, alkenyl group, alkynyl group or aralkyl group is the alkylating agent of the Formula (V)

RX (V)

wherein R is the R³ substituent of the end-product as defined above, X is a leaving group, e.g. halogen atom, methanesulfonyloxy group or p-toluenesulfonyloxy group, preferably a halogen atom, more preferably a chlorine, bromine, or iodine atom. The base used is an organic or inorganic base, e.g. alkali metal carbonate, alkali metal hydrogencarbonate, aliphatic or alicyclic secondary or tertiary amines, preferably 1,8-diazabicyclo[5.4.0]undec-7-ene (DBU).

If desired, the 2,3,4-benzothiadiazepine-2,2-dioxide derivatives of Formula (I), wherein R³ represents alkyl group, alkenyl group or aralkyl group and R⁴ represents hydrogen, can be converted to compounds of Formula (I), wherein R³ and R⁴ represents alkyl group, alkenyl group, alkynyl group or aralkyl group, by reacting with an alkylating agent of the Formula (V), wherein R represents R⁴ substituent of the end-product as defined herein above, in the presence of a strong base. The strong base is an organic or inorganic base, e.g. alkali metal alkoxide, alkali metal hydride, alkaline earth metal hydride or alkyllithium compound, preferably alkyl lithium compound, more preferably butyllithium.

There is a one-pot method, wherein the 2,3,4-benzothiadiazepine-2,2-dioxide derivatives of Formula (I), wherein R³ and R⁴ represents hydrogen can be converted directly to compounds of Formula (I) wherein R³ and R⁴ represent the same alkyl group, alkenyl group, alkynyl group or aralkyl group by reacting with alkylating agent of the Formula (V) in the presence of a strong base.

In this case the alkylating agent of the Formula (V) as defined above is used in 2 molar equivalent amount relative to the molar amount of the starting compound. The strong base in the alkylation reaction is organic or inorganic base in 2 molar equivalent amount, e.g. alkali metal alkoxide, alkali metal hydride, alkaline earth metal hydride or alkyl lithium compound, preferably alkyllithium compound, more preferably butyllithium.

It is known from the literature that taurine, which is a non-proteinaceous amino acid, occurs in high quantities in the central nervous system (Oja and Saransaari, Proc. West. Pharmacol. Soc., 50: 8-15, 2007). Taurine plays important role in several neurophysiologic processes. It is also known that taurine possesses antioxidant properties and regulates the intracellular calcium level, thus modulates neuronal receptor function (Szymansky and Winiarska, Postepy. Hlg. Med. Dosw., 62: 75-86, 2008). The extent of long-term potentiation (LTP) in striatum decreased in taurine transporter knock-out mice and there were pathological changes in neuroreceptor expression (Warskulat and coworkers, Methods Enzymol., 428: 439-58, 2007). These results support the important modulator role of taurine in the central nervous system.

On the basis of above mentioned information and the test data herein that we obtained for the compound of the Formula (I) when administered to rats where we determined the taurine concentrations measured in rats' frontal cortex after administration of the Formula (I) compounds, we have provided evidence that one may use the new compounds of the Formula (I) for the treatment of different diseases with neurodegenerative origin, including Alzheimer's disease, Huntington's disease, Parkinson disease etc. Taurine also stimulates the cognitive processes (Seidl R. et al, Amino Acids, 19: 635-642, 2000). The compounds of the Formula (I) according to the invention which elevate the taurine level in the cortex furthermore have therapeutic effects in diseases affecting the central nervous system marked by cognitive decline including schizophrenia, vascular dementia, post-stroke dementia, senile dementia not of the Alzheimer's type, Down's syndrome, Korsakoff syndrome. The anxiolytic/antistress effect of taurine also was published (Kong W X. et al, Pharmacol Biochem Behaviour, 83: 271-276, 2006). The beneficial taurine increasing effect of compounds of the Formula (I) of the present application include anxiolytic and antidepressant properties of these molecules and using them for the therapy of generalized anxiety disorder, panic disorder, posttraumatic stress disorder. and different forms of depressions, including monopolar depression and bipolar depression.

A further feature of the present invention are pharmaceutical compositions comprising as active ingredient one or more compounds of the Formula (I) as defined herein above and/or enantiomers and/or pharmaceutically acceptable acid-addition salts thereof in a therapeutically effective dose together with one or more pharmaceutically applicable diluent, excipient and/or inert carrier.

The pharmaceutical compositions of the present invention contain in general 0.1 to 95.0 percent by weight, preferably 1.0 to 50.0 percent by mass, more preferably 5.0 to 30.0 percent by mass of active ingredient.

The pharmaceutical compositions of the present invention are suitable for oral (e.g. powders, tablets, film-coated tablets, capsules, microcapsules, drops/pills, solutions, suspensions or emulsions), parenteral (e.g. in form of intravenous, intramuscular, subcutaneous or intraperitoneal injections or infusion compositions), rectal (e.g. in form of suppositories), transdermal (e.g. patches) administration, or can be used in the form of implants or for local treatment (e.g. ointment, gels or patches).

The solid, semisolid or liquid pharmaceutical dosage forms of the present invention are known per se, the methods of manufacturing the compositions are known in the state of the art.

The solid pharmaceutical compositions suitable for oral administration may be powders, capsules, tablets, film-coated tablets, microcapsules etc., and optionally comprise binding agents such as gelatine, sorbitol, polyvinylpyrrolidone) etc., filling agents such as lactose, glucose, starch, calcium phosphate etc., auxiliary substances for tabletting such as magnesium stearate, talc, polyethylene glycol, silica etc., wetting agents such as sodium laurylsulfate etc.

The liquid pharmaceutical compositions suitable for oral administration may be solutions, suspensions or emulsions and can comprise e.g. suspending agents such as gelatine, carboxymethyl cellulose etc., emulsifiers such as sorbitane monooleate etc., solvents such as water, oils, glycerol, propyleneglycol, ethanol etc., preservatives such as methy p-hydroxybenzoate etc., and the carrier.

Typical parenteral compositions consisting of a solution or suspension of the compound of Formula (I) and/or enantiomers and/or pharmaceutically suitable acid additional salts thereof in a sterile aqueous carrier or parenterally acceptable oil, for example polyethylene glycol, polyvinyl pyrrohdone, lecithin, arachis oil or sesame oil. Alternatively, the solution can be lyophilised and then reconstituted with a suitable solvent just prior to adiriinistration.

Compositions of the present invention for nasal administration containing a compound of Formula (I) and/or enantiomers and/or pharmaceutically suitable acid additional salts thereof may conveniently be formulated as aerosols, drops, gels and powders.

Aerosol formulations of the present invention typically comprise a solution or fine suspension of the compound of Formula (I) and/or enantiomers and/or pharmaceutically suitable acid additional salts thereof in a physiologically acceptable aqueous or non-aqueous solvent and are usually presented in a single or multidose quantities in sterile form in a sealed container, which can take the form of a cartridge or refill for use with an atomizing device. Alternatively, the sealed container may be a unitary dispensing device, such as a single dose nasal inhaler or an aerosol dispenser fitted with a metering valve which is intended for disposal once the contents of the container have been exhausted. Where the dosage form comprises an aerosol dispenser, it will contain a propellant which can be a compressed gas, such as compressed air or an organic propellant, such as a fluorochlorohydrocarbon. The adniinistration of aerosol dosages can also take the form of a pump-atomiser.

Compositions of the present invention containing a compound of Formula (I) and/or enantiomers and/or pharmaceutically suitable acid additional salts thereof are suitable for buccal or sublingual administration including tablets, lozenges and pastilles, wherein the active ingredient is formulated with a carrier, such as sugar and acacia, tragacanth, or gelatine, glycerol, etc.

Compositions of the present invention containing a compound of Formula (I) and/or enantiomers and/or pharmaceutically suitable acid additional salts thereof for rectal administration are conveniently in the form of suppositories containing a conventional suppository base, such as cocoa butter, polyethylene glycol or other materials commonly used in the art. The suppositories may be conveniently formed by first admixing the composition with the softened or melted carrier(s) followed by chilling and shaping in moulds.

Compositions of the present invention containing a compound of Formula (I) and/or pharmaceutically suitable acid additional salts thereof for transdermal administration include ointments, gels and patches.

Dosage forms listed above as well as other dosage forms are known per se, see e.g. Remington's Pharmaceutical Sciences, 18^{th} Edition, Mack Publishing Co., Easton, USA (1990).

The compositions containing the compounds of the Formula (I) of the present inventions contain the active ingredient in form of dose units.

The typical dosage for adult patients (e.g. adults weighing 60 to 80 kg) is 0.1-1000 mg of a compound of Formula (I) or an enantiomer or a pharmaceutically acceptable acid additional salt thereof, which can be administered once or in portions. The actual dose depends on many factors and its determination of is a medical task.

A further feature of the invention is the medical use of the compounds of the Formula (I) and or enantiomers and/or pharmaceutically suitable acid additional salts thereof as a medicament in the treatment of diseases of the central nervous system. Furthermore our invention relates to a method of treatment, which comprises administering a patient suffering from a central nervous system disorder a therapeutically effective dose of one or more compounds of Formula (I) as defined in claim 1 and/or enantiomers and/or pharmaceutically suitable acid additional salts thereof.

In the investigations of biological efficiency of the compounds of the Formula (I) as defined above, it has been found that the compounds are effectively modulating central nervous system functions, thus, the compounds of the Formula (I) according to the invention are well suited for the prevention and/or treatment of several diseases of the central nervous system.

The in vivo biological effectiveness of the compounds of the present invention on the central nervous system was examined by the following experimental model.

Changes in Amino Acid Neurotransmitter Levels in the Cortical Area of Male Rats

Male SPDR rats weighing 200-250 g, were used in the experiments. Changes in the concentration of amino acid neurotransmitters were measured from cortical areas by high-performance liquid chromatography (HPLC) and electrochemical detection.

The investigations were carried out with two experimental groups wherein each group contained 8 animals. One group was administered intraperitoneally by vehicle only (control group). The other group was administered intraperitoneally by the compound of the Example 15, suspended in vehicle. The dose of administration was in the form of 0.4% aqueous methylcellulose suspension.

The animals were decapitated 30 minutes after the administration, brains have been removed and required brain regions were dissected. The tissues were stored at −70° C.

The tissues were homogenized in ice-cold 0.1 M perchlorate in 500 volume excess. The homogenates were intensively mixed, centrifuged at 4° C. at 20,000 rpm for 20 minutes, the supernatants were removed and an aliquot thereof was immediately injected after derivatisation without further sample purification.

The automatic derivatisation was carried out at 25° C. using o-phthalaldehyde/Na₂S0₃ reagent; the reaction time was 10 minutes.

The assay was performed using an Agilent 1100 HPLC, separation was carried out by gradient elution on an Agilent Zorbax SB-C18 (250×4.6 mm, 5μπι) column, utilising 0.1 M NaH₂P0₄, pH=4.5/methanol eluent.

For electrochemical detection, ESA Coulochem III detector was used (analytical cell: ESA 5011 A), the potential of working electrode was +650 mV.

The tissue concentrations of the amino acids were measured by standard solutions containing amino acids with known concentration.

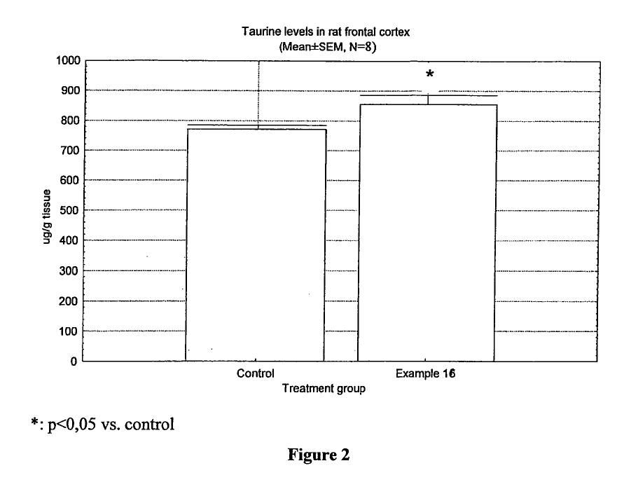

It has been found that concentrations of the amino acid taurine were significantly increased in the frontal cortex of the animals administered with the compound of Example 16 of the present invention, compared to frontal cortex obtained from animals of the control group. FIG. 2 demonstrates the results.

Again, it is known from the literature that taurine, which is non-proteinaceous amino acid, occurs in high quantities in the central nervous system (Oja and Saransaari, Proc. West. Pharmacol. Soc, 50i 8-15, 2007). Taurine plays important role in several neurophysiologic processes. It is also known that taurine possesses antioxidant properties and regulates the intracellular calcium level, thus modulates neuronal receptor function (Szymansk and Winiarska, Postepy. Hig. Med. Dosw., 62_{—}75-86, 2008). The extent of long-term potentiation (LTP) in striatum decreased in taurine transporter knock-out mice and there were pathological changes in neuroreceptor expression (Warskulat is mtsai, Methods Enzymol, 428: 439-58, 2007). These results support the important modulator role of taurine in the central nervous system.

It has been surprisingly found that compounds of the present invention induce a statistically significant increase in the amount of taurine in the examined brain regions, resulting in the effective modulation of neuronal functions by the taurine pathway.

Therefore, the compounds of the Formula (I) of the present invention are well suited for the prevention and/or treatment of several diseases of the central nervous system. Due to this surprising effect, it has been recognized that the compounds of the present invention are able to modulate the active function of central nervous system, normalize the pathological features, thus are expected to be effective in the treatment of disorders of the central nervous system, including Alzheimer's disease, Huntington's disease, Parkinson disease, schizophrenia, vascular dementia, post-stroke dementia, senile dementia not of the Alzheimer's type, Down's syndrome, Korsakoff' syndrome. Furthermore the compounds of the Formula (I) exert anxiolytic and antidepressant effects and may be used for the therapy of Generalized Anxiety disorder, Panic disorder, Posttraumatic Stress disorder. and different forms of depressions, including monopolar depression and bipolar depression.

The invention is further elucidated by means of following Examples without restricting the scope of the present invention to the Examples.

### EXAMPLES

### Halogen-Sulfite Exchange (Process “A”)

To the solution of 540 ml dioxane and 800 ml water, 0.27 mol of (substituted 2-chloromethyl-phenyl)-(substituted aryl)-methanone and 0.36 mol of sodium sulfite are added and boiled for ca. two hours. After cooling ca. 800 ml solvent is evaporated from the reaction mixture.

Process A/1: the residual aqueous phase is extracted with ethyl acetate or tert-butyl methyl ether and the organic phase is extracted with 2×80 ml 1M NaOH solution. The combined alkaline and aqueous phase is decoloured with activated carbon and evaporated to ca. 250 ml. Thus an oily product is obtained. 52 ml of concentrated hydrochloric acid is added to the mixture under cooling and stirring and the reaction mixture is stirred for half an hour. The precipitated substance is filtered, washed and dried (sodium salt).

Process A 2: If the evaporation of the aqueous phase results in sulfonic acid crystals, the crystals are filtered, washed and dried.

### Example 1

### Sodium [4-chloro-2-(4-fluoro-benzoyl)-phenyl]-methanesulfonate

The title product is prepared according to the process A/1 starting from (5-chloro-2-chloromethyl-phenyl)-(4-fluorophenyl)-methanone.

Yield: 60%

Melting point: 254-256° C. (water)

IR (KBr): 3448, 1667, 1598, 1412 cm^{″1}

HNMR (DMSO-t¾, 500 MHz): 7.80 (dd, J=8.8, 5.7 Hz, 2H), 7.55 (m, 2H), 7.35 (t, J=8.8 Hz, 2H), 7.23 (m, 1H), 3.98 (s, 2H) ppm.

CNMR (DMSO-i/₆, 125 MHz): 194.9, 165.1 (d, J=251.5 Hz), 141.0, 134.6, 134.0 (d, J=2.9 Hz), 133.6, 133.4 (d, J=9.3 Hz), 130.8, 129.9, 127.9, 115.5 (d, J=22.0 Hz), 53.0 ppm.

Elemental analysis [calculated on the basis of the Formula C) 4H₉ClF0₄SNa (350.73)]

Calculated: C, 47.94; H, 2.59; CI, 10.11; S, 9.14%.

Measured: C, 47.56; H, 2.77; CI, 10.00; S 9.06%.

Gravimetric Na content: 6.22% (calculated: 6.55%)

### Example 2

### Sodium [4,5-dichloro-2-(4-fluoro-benzoyl)-phenyl]-methanesulfonate

The title product is prepared according to the process A/1 starting from (4,5-dichloro-2-chloromethyl-phenyl)-(4-fluorophenyl)-methanone.

Yield: 70%

Melting point: 216-230° C. (water)

IR (KBr): 3456, 1758, 1667, 1598, 1506, 1285, 1225, 1149, 1044 cm^{″1}.

HNMR (DMSO-<¾, 500 MHz): 7.79 (dd, J=8.8, 5.6 Hz, 2H), 7.75 (s, 1H), 7.46 (s, 1H), 7.35 (t, J=8.9 Hz, 2H), 3.96 (s, 2H) ppm.

### Example 3

### Sodium [6-[(3,4-dimethoxyphenyl)-carbonyl]-2,1₅3-benzothiadiazoI-5-yl]-methanesulfonate

The title product is prepared according to the process A/2 starting from [6-(chloromethyl)-2,1,3-benzothiadiazol-5-yl](3,4-dimethoxyphenyl)-methanone.

Yield: 86%

Melting point: 282-283° C. (water)

HNMR (DMSO-ti₆, 500 MHz): 8.15 (d, J=0.6 Hz, 1H), 7.91 (d, J=0.5 Hz, 1H), 7.49 (d, J=2.0 Hz, 1H), 7.36 (dd, J=2.0, 8.4 Hz, 1H), 7.07 (d, J=8.5 Hz, 1H), 4.13 (s, 2H), 3.87 (s, 3H), 3.81 (s, 3H) ppm.

### Example 4

### Sodium [6-[(4-fluorophenyl)-carbonyl]-2,1,3-benzothiadiazol-5-yl]-methanesulfonate

The title product is prepared according to the process A/2 starting from [6-(chloromethyl)-2,1,3-benzothiadiazol-5-yl] (4-methoxyphenyl)-methanone.

Yield: 90%

Melting point: 320-323° C. (water), with decomposition

IR (KBr): 3527, 1657, 1600, 1207, 1044 cm⁻¹.

HNMR (DMSO-<¼, 500 MHz): 8.09 (d, J=0.6 Hz, 1H), 7.91 (d, J=0.6 Hz, 1H), 7.90 (dd, J=8.8, 5.5 Hz, 2H), 7.36 (t, J=8.9 Hz, 2H), 4.21 (s, 2H) ppm.

CNMR (DMSO-i₆, 125 MHz): 194.89, 165.10 (d, J=251.5 Hz), 154.62, 152.35, 141.24, 136.54, 134.03 (d, J=2.9 Hz), 133.76 (d, J=9.3 Hz), 124.14, 121.50, 115.32 (d, J=22.0 Hz), 53.74 ppm.

Elemental analysis [calculated on the basis of the Formula C₁₄H₈FN₂O₄S₂Na (374.35)]

Calculated: C, 44.92; H, 2.15; N, 7.48%.

Measured: C, 44.59; H, 2.16; N, 7.23%.

### Example 5

### Sodium [6-[(4-methoxyphenyl)-carbonyl]-2,1,3-benzothiadiazol-5-yl]-methanesulfonate

The title product is prepared according to the process A/2 starting from [6-(chloromethyl)-2,1,3-benzothiadiazol-5-yl](4-methoxyphenyl)methanone

Yield: 79%

Melting point: 287-288° C. (water), with decomposition

IR (KBr): 3527, 1647, 1598, 1300 cm^{″1}.

HNMR (DMSO-i4, 125 MHz): 8.13 (s, 1H), 7.89 (s, 1H), 7.80 (d, J=9.2 Hz, 2H), 7.06 (d, J=9.2 Hz, 2H), 4.16 (s, 2H), 3.87 (s, 3H) ppm.

Ring closing reaction (process “B”)

78 mmol of sodium [2-(substituted-benzoyl)-substituted phenyl]-methanesulfonate and 100 ml thionyl chloride are boiled for 4 hours. The reaction mixture is evaporated. The residue is dissolved in 250 ml of dichloromethane and the solution is added dropwise to the solution of 750 ml dichloromethane and 75 ml hydrazine under cooling and vigorous stirring. The mixture is stirred for 1.5 hours without cooling. The solution is acidified under ice-cooling with 160 ml cc. hydrochloric acid (to pH=1). The phases are separated and the aqueous phase is extracted with 2×100 ml dichloromethane. The combined aqueous layer is dried over MgSO₄ and evaporated.

Process B/1: the crystalline residue is triturated with mixture of 40 ml ethanol and 30 ml hexane, filtered, washed with 2×20 ml cold ethanol, and dried.

Process B/2: the residue is subjected to flash chromatographic purification with the appropriate solvent and the product is obtained by evaporation.

### Example 6

### 8-Cyano-5-(4-fluoro-phenyl)-1,3-dihydro-2,3,4-benzothiadiazepin-2,2-dioxide

The title product is prepared according to the process B/1 starting from sodium [5-cyano-2-(4-fluoro-benzoyl)-phenyl]-methanesulfonate.

Yield: 35%

Melting point: 218-219° C. (ethanol)

IR (KBr): 3215, 1601, 1510, 1325, 1150 cm-¹.

HNMR (DMSO-<, 200 MHz): 10.52 (bs, IH), 8.16 (d, J=1.5 Hz, 1H), 7.96 (dd, J=8.2, 1.8 Hz, IH), 7.67 (dd, J=9.2, 5.8 Hz, 2H), 7.44 (d, J=7.9 Hz, IH), 7.34 (t, J=8.9 Hz, 2H), 4.83 (s, 2H) ppm.

Elemental analysis [calculated on the basis of the Formula [C₁₅H₁₀FN₃O₂S

(315.33)]

Calculated: C, 57.14; H, 3.20; N, 13.30%.

Measured: C, 57.89; H, 3.39; N, 13.29%.

### Example 7

### 7-Chloro-5-(3-fluoro-phenyl)-1,3-dihydro-2,3,4-benzothiadiazepin-2,2-dioxide

The title product is prepared according to the process B/1 starting from sodium [4-chloro-2-(3-fluoro-benzoyl)-phenyl]-methanesulfonate.

Yield: 42%

Melting point: 195-198° C. (ethanol)

IR (KBr): 3200, 1320, 1159 cm⁻¹.

HNMR (CDCI3, 200 MHz): 7.58 (dd, J=8.1, 2.2 Hz, 1H), 7.46 (d, J=8.4 Hz, 1H), 7.19-7.50 (m, 5H), 7.14 (bs, 1H), 4.40 (s, 2H) ppm.

Elemental analysis [calculated on the basis of the Formula C₁₄H₁₀ClFN₂O₂S

(324.76)]

Calculated: C, 51.78; H, 3.10; CI, 10.92; N, 8.63%.

Measured: C, 52.13; H, 3.09; CI, 10.85; N, 8.44%.

### Example 8

### 9-(4-Methylphenyl)-5H,7H-[1,2,5]thiadiazolo[3,4-h]-2,3,4-benzothiadiazepin-6,6-dioxide

The title product is prepared according to the process B/1 starting from sodium [6-[(4-methoxyphenyl)-carbonyl]-2,1,3-benzothiadiazol-5-yl]-methanesulfonate.

Yield: 70%

Melting point: 212-215° C. (ethyl acetate)

IR (KBr): 3095, 1325, 1150 cm-¹.

HNMR (CDCI₃, 125 MHz): 10.26 (s, 1H), 8.38 (d, J=0.6 Hz, 1H), 7.95 (s, 1H), 7.62 (d, J=8.2, 2H), 7.32 (d, J=8.1 Hz, 2H), 4.92 (s, 2H), 2.39 (s, 3H) ppm.

Elemental analysis [calculated on the basis of the Formula C₁₅H₁₂N₄0₂S₂

(344.42)]

Calculated: C, 52.31; H, 3.51; N, 16.27%.

Measured: C, 51.98; H, 3.45; N, 16.32%.

### Example 9

### 9-(4-Methoxyphenyl)-5H,7H-[1,2,5]thiadiazoIo[3,4-h][2,3,4]benzothiadiazepin-6,6-dioxide

The title product is prepared according to the process B/1 starting from sodium 46-(4-methoxy-berzoyl)-benzo[1,2,5]thiadiazol-5-yl]-methanesulfonate.

Yield: 68%

Melting point: 225-227° C. (ethyl acetate)

IR (KBr): 3101, 1601, 1321, 1148 cm^{″1}.

HNMR (CDCl₃, 200 MHz): 10.19 (bs, 1H), 8.37 (s, 1H), 7.98 (s, 1H), 7.70 (d, J=8.8 Hz, 2H), 7.05 (d, J=8.8 Hz, 2H), 4.92 (s, 2H), 3.84 (s, 3H) ppm.

Elemental analysis [calculated on the basis of the Formula C₁₅H₁₂N₄0₃S₂

(360.42)]

Calculated: C, 49.99; H, 3.36; N, 15.55%.

Measured: C, 49.11; H, 3.45; N, 15.29%.

### Example 10

### 9-(3,4-Dimethoxyphenyl)-5H,7H-[1,2,5]thiadiazolo[3,4-h][2,3,4]benzothiadiazepin-6,6-dioxide

The title product is prepared according to the process B/1 starting from sodium [6-(3,4-dimethoxy-benzoyl)-benzo[1,2,5]thiadiazoI-5-yl]-methanesulfonate.

Yield: 70%

Melting point: 228-230° C. (ethyl acetate)

IR (KBr): 3096, 1517, 1322 cm⁻¹.

HNMR (CDCl₃, 125 MHz): 10.16 (s, 1H), 8.37 (s, 1H), 7.93 (s, IK), 7.52 (s, IK), 7.04 (m, 2H), 4.89 (s, 2H), 3.83 (s, 3H), 3.81 (bs, 6H) ppm.

Elemental analysis [calculated on the basis of the Formula C₁₆H₁₄N₄0₄S2

(390.44)]

Calculated: C, 49.22; H, 3.61; N, 14.35%.

Measured: C, 49.00; H, 3.72; N, 14.12%.

### Example 11

### 9-(4-FIuorophenyl)-5H′,7̂-[1,2,5]thiadiazolo[3,4-A][2,3,4]beiLzothiadiazepin-6,6-dioxide

The title product is prepared according to the process B/1 starting from sodium [6-[(4-fluorophenyl)-carbonyl]-2,1,3-benzothiadiazol-5-yl]-methanesulfonate.

Yield: 68%

Melting point: 222-225° C. (ethanol-DMF)

IR (KBr): 3137, 1597, 1569, 1508, 1326 cm^{″1}.

HNMR (DMSO-ii₆, 500 MHz): 10.34 (s, 1H), 8.38 (s, 1H), 8.02 (s, 1H), 7.80 (dd, J=8.7, 5.5 Hz, 2H), 7.36 (t, J=8.9 Hz, 2H), 4.96 (s, 2H) ppm.

C MR (DMSO-</₆, 125 MHz): 172.04, 164.33 (d, J=250.0 Hz), 154.43, 152.96, 136.26, 133.11, 132.42 (d, J=2.9 Hz), 132.19 (d, J=8.7 Hz), 122.60, 121.47, 115.81 (d, J=22.0 Hz), 55.10 ppm.

Elemental analysis [calculated on the basis of the Formula Ci₄H₉FN₄0₄S₂

(348.38)]

Calculated: C, 48.27; H, 2.60; N, 16.08; S, 18.41%.

Measured: C, 48.30; H, 2.55; N, 16.17; S, 18.43%.

### Example 12

### 7-Chloro-5-(4-fluoro-phenyl)-1,3-dihydro-2,3,4-benzothiadiazepin-2,2-dioxide

The title product is prepared according to the process B/1 starting from sodium [4-chloro-2-(4-fluoro-benzoyl)-phenyl]-methanesulfonate.

Yield: 68%

Melting point: 195-198° C. (methanol)

IR (KBr): 3222, 1602, 1508, 1329, 1157 cm^{″1}.

HNMR (CDCI3, 500 MHz): 7.70 (dd, J=5.3 Hz, 2H), 7.57 (dd, J=8.2, 2.1 Hz, 1H), 7.46 (d, J=8.2 Hz, 1H), 7.25 (d, J=2.2 Hz, 1H), 7.15 (t, J=8.6 Hz, 2H), 7.08 (bs, 1H), 4.39 (s, 2H) ppm.

CNMR (CDCI3, 125 MHz): 171.2, 165.1 (d, J=254.4 Hz), 135.3, 135.0, 131.7 (d, J=8.8 Hz), 131.7, 131.0 (d, J=2.9 Hz), 130.7, 129.5, 129.2, 116.0 (d, J=22.0 Hz), 55.7 ppm.

Elemental analysis [calculated on the basis of the Formula Ci4H₁₀ClFN2O2S (324.76)]

Calculated: C, 51.78; H, 3.10; N, 8.63%.

Measured: C, 51.61; H, 3.16; N, 8.71%.

### Example 13

### 7-Fluoro-5-(4-fluoro-phenyl)-1,3-dihydro-2,3,4-benzothiadiazepin-2,2-dioxide

The title product is prepared according to the process B/1 starting from, sodium [4-fluoro-2-(4-fluoro-benzoyl)-phenyl]-methanesulfonate.

Yield: 62%

Melting point: 196-200° C. (ethanol-hexane)

IR (KBr): 3201, 1602, 1507, 1318, 1163 cm^{′1}.

HNMR (DMSO-fife, 500 MHz): 10.33 (s, 1H), 7.68 (m, 3H), 7.49 (td, J=8.5, 2.7 Hz, 1H), 7.34 (t, J=8.8 Hz, 2H), 7.10 (dd, J=9.2, 2.7 Hz, 1H), 4.71 (s, 2H) ppm. CNMR (DMSO-<&, 125 MHz): 170.4 (d, J=1.5 Hz), 164.1 (d, J=249.5 Hz), 161.2 (d, J=246.6 Hz), 136.1 (d, J=7.8 Hz), 132.2 (d, J=2.9 Hz), 131.9 (d, J=8.8 Hz), 131.8 (d, J=8.8 Hz), 128.9 (d, J=3.4 Hz), 118.2 (d, J=22.0 Hz), 115.9 (d, J=23.4 Hz), 115.8 (d, J=22.0 Hz), 54.6 ppm.

Elemental analysis [calculated on the basis of the Formula Ci₄H₁₀F₂N₂O₂S (308.31)]

Calculated: C, 54.54; H, 3.27; S, 10.40; N, 9.09%.

Measured: C, 54.89; H, 3.23; S, 10.28; N, 9.16%.

### Example 14

### 7,8-Dichloro-5-(4-fluoro-phenyl)-1,3-dihydro-2,3,4-benzothiadiazepin-2,2-dioxide

The title product is prepared according to the process B/1 starting from sodium [4,5-dichloro-2-(4-fluoro-benzoyl)-phenyl]-methanesulfonate.

Yield: 72%

Melting point: 196-200° C. (acetonitrile)

IR (KBr): 3443, 3200, 1599, 1508, 1327, 1166 cm^{′1}.

HNMR (DMSO-fife, 500 MHz): 10.45 (s, 1H), 8.00 (s, 1H), 7.69 (dd, J=9.0, 5.5 Hz, 2H), 7.51 (s, 1H), 7.35 (t, J=8.8 Hz, 2H), 4.77 (s, 2H) ppm.

CNMR (DMSO-</₆, 125 MHz): 169.9, 164.2 (d, J=250 Hz), 134.5, 133.8, 133.0, 132.0 (d, J=8.8 Hz), 131.9 (d, J=2.9 Hz), 131.2, 131.1, 130.8 (d, J=22.0 Hz), 54.4 ppm.

Elemental analysis [calculated on the basis of the Formula Ci4H9Cl₂FN₂0₂S

(359.21)]

Calculated: C, 46.81; H, 2.53; S, 8.93; CI, 19.74; N, 7.80%.

Measured: C, 46.55; H, 2.63; S, 8.78; CI, 19.51; N, 7.71%.

### Example 15

### 6-{[(4-Fluorophenyl)-carbonyl]-2,1-benzothiadiazol-5-yl}-methanesulfonic acid chloride

5.34 mmol sodium [6-[(4-fluorophenyl)-carbonyl]-2,1,3-benzothiadiazol-5-yl]-methanesulfonate is added to 5 ml thionyl chloride and are boiled for 2.5 hours. After cooling the reaction mixture is evaporated. The residue is dissolved in 10 ml chloroform and is clarified by carbon, after filtering the mixture is evaporated. The residue is triturated with hexane, filtered and dried.

Yield: 85%

Melting point: 148-151° C. (hexane)

IR (KBr): 2993, 1665, 1595, 1230, 850 cm^{″1}.

HNMR (CDCl₃, 500 MHz): 8.33 (s, 1H), 8.17 (s, 1H), 7.95 (dd, J=5.4, 3.5 Hz, 2H), 7.21 (t, J=8.5 Hz, 2H), 5.60 (s, 2H) ppm.

CNMR (CDCI3, 125 MHz): 194.6, 166.10 (d, J=256.8 Hz), 154.4, 153.6, 138.3, 133.5, 133.4, 132.7 (d, J=3.0 Hz), 128.1, 127.0, 125.0, 115.9, 77.0 (t, J=31.7 Hz), 66.7 ppm.

Elemental analysis [calculated on the basis of the Formula C₁₄H₈ClFN₂0₃S₂

(370.81)]

Calculated: C, 45.35; H, 2.17; N, 7.55%.

Measured: C, 45.59; H, 2.16; N, 7.23%.

N(3)-alkylating reaction (Process “C”)

119 mmol of 1,3-dihydro-2,3,4-berizothiadiazepin-2,2-dioxide, appropriately substituted on the benzene ring and containing an appropriately substituted phenyl group in position 5, is added to 1000 ml acetonitrile. 21.45 ml (144 mmol) of DBU and 146 mmol alkylating agent of the Formula (V) are added to the solution at room temperature. The reaction mixture is stirred at 20-60° C. until the starting material is used up. The amount of starting material is monitored by TLC.

The reaction mixture is evaporated and dissolved in 300 ml dichloromethane and 250 ml water. After separating, the aqueous layer is extracted with 100 ml dichloromethane. The combined organic phase is washed with 2×200 ml water, dried over MgS0₄ and evaporated.

Process C/1: the crystalline residue is triturated with 50 ml methanol and after stirring for 30 min, it is filtered and dried. An analytical sample is obtained by recrystallization from the solvent indicated.

Process C/2: If the residue is oily and not crystallized from methanol, the residue is subjected to flash chromatography on silica gel (20-fold amount) with hexane; ethyl acetate=1:1 eluent and it is crystallised. The solvent used for recrystallization is indicated right after the melting point of each compound.

Process C/3: if the residue is crystalline but soluble in methanol the residue is crystallized. The solvent used for recrystallization is indicated right after the melting point of each compound.

### Example 16

### 7-Chloro-5-(4-fluoro-phenyl)-3-methyl-1,3-dihydro-2̂,4-benzothiadiazepin-2,2-dioxide

The title product is prepared according to the process C/1 starting from, 7-chloro-5-(4-fluoro-phenyl)-1,3-dmydro-2,3,4-benzothiadiazepin-2,2-dioxide and methyl iodide.

Yield: 92%

Melting point: 219-221° C. (acetonitrile)

IR (KBr): 2966, 2944, 1601, 1509, 1323, 1135 cm^{″1}.

HNMR (CDCI3, 500 MHz): 7.71 (dd, J=9.0, 5.4 Hz, 2H), 7.55 (dd, J=8.3, 2.2 Hz, 1H), 7.45 (d, J=8.2 Hz, 1H), 7.24 (d, J=2.2 Hz, IH), 7.14 (t, J=8.6 Hz, 2H), 4.35 (s, 2H), 3.49 (s, 3H) ppm.

CNMR (CDCI3, 125 MHz): 169.9, 165.1 (d, J=253.9 Hz), 135.7, 134.9, 131.7 (d, J=8.8 Hz), 131.6, 130.9 (d, J=3.4 Hz), 130.4, 128.9, 128.7, 116.0 (d, J=22.0 Hz), 55.3, 35.3 ppm.

Elemental analysis [calculated on the basis of the Formula C₁₅H₁₂ClFN₂O₂S (338.79)

Calculated: C, 53.18; H, 3.57; N, 8.27; S, 9.46; CI, 10.46%.

Measured: C, 52.96; H, 3.56; N, 8.23; S, 9.55; CI, 10.53%.

### Example 17

### 7-Chloro-3-ethyl-5-(4-fluoro-phenyl)-1,3-dihydro-2,3,4-benzothiadiazepin-2,2-dioxide

The title product is prepared according to the process C/1 starting from 7-chloro-5-(4-fluoro-phenyl)-1,3-dihydro-2,3,4-benzothiadiazepin-2,2-dioxide and ethyl iodide.

Yield: 90%

Melting point: 171-172° C. (diisopropyl ether-ethanol)

IR (KBr): 2976, 1603, 1508, 1326, 1137 cm^{′1}.

HNMR (DMSO-<4, 500 MHz): 7.72 (dd, J=8.3, 2.2 Hz, 1H), 7.71 (dd, J=8.9, 5.4 Hz, 2H), 7.70 (d, J=8.4 Hz, 1H), 7.36 (t, J=8.8 Hz, 2H), 7.31 (d, J=2.0 Hz, 1H), 4.84 (s, 2H), 3.45 (q, 2H), 1.05 (t, 3H) ppm.

CNMR (DMSO-d₆, 125 MHz): 170.5, 164.3 (d, J=250.0 Hz), 136.1, 133.1, 132.1 (d, J=8.8 Hz), 131.8 (d, J=2.9 Hz), 131.4, 131.4, 130.6, 128.1, 115.9 (d, J=22.0 Hz), 54.3, 42.4, 13.1 ppm.

Elemental analysis [calculated on the basis of the Formula C₁₆H₁₄ClFN₂0₂S (352.82)]

Calculated: C, 54.47; H, 4.00; CI, 10.05; S, 9.09; N, 7.94%.

Measured: C, 54.32; H, 3.92; CI 9.87; S, 9.29; N, 7.93%.

### Example 18

### 7-Chloro-5-(4-fluoro-phenyl)-3-propyl-1,3-dihydro-2,3,4-benzothiadiazepin-2,2-dioxide

The title product is prepared according to the process C/2 starting from 7-chloro-5-(4-fluoro-phenyl)-1,3-dihydro-2,3,4-benzothiadiazepin-2,2-dioxide and propyl iodide.

Yield: 85%

Melting point: 122-122.5° C. (diisopropyl ether-hexane)

IR (KBr): 2938, 1601, 1509, 1335, 1135 cm-¹.

HNMR (CDCl₃, 500 MHz): 7.72 (dd, J=8.9, 5.4 Hz, 2H), 7.55 (dd, J=8.2, 2.2 Hz, 1H), 7.45 (d, J=8.3 Hz, 1H), 7.22 (d, J=2.1 Hz, 1H), 7.14 (t, J=8.7 Hz, 2H), 4.34 (s, 2H), 3.53 (t, J=7.1 Hz, 2H), 1.49 (m, 2H), 0.72 (t, J=7.4 Hz, 3H) ppm.

CNMR (CDCI3, 125 MHz): 170.9, 165.0 (d, J=254.4 Hz), 135.9, 134.6, 131.6 (d, J=9.3 Hz), 131.4, 131.0 (d, J=2.9 Hz), 130.4, 129.3, 128.2, 115.9 (d, J=22.0 Hz), 55.5, 49.2, 21.1, 10.8 ppm.

Elemental analysis [calculated on the basis of the Formula C₁₇H₁₆ClFN₂0₂S (366.84)]

Calculated: C, 55.66; H, 4.40; CI, 9.66; S, 8.74; N, 7.64%.

Measured: C, 55.68; H, 4.41; CI, 9.62; S, 8.82; N, 7.57%.

### Example 19

### 7-Chloro-5-(4-fluoro-phenyl)-3-(propynyl)-1,3-dihydro-2,3,4-benzothiadiazepin-2,2-dioxide

The title product is prepared according to the process C/2 starting from 7-chloro-5-(4-fluoro-phenyl)-1,3-dihydro-2,3,4-benzothiadiazepin-2,2-dioxide and 3-bromo-propyne.

Yield: 91%

Melting point: 139-140° C. (diisopropyl ether)

IR (KBr): 3305, 1605, 1508, 1329, 1131, 1065, 844 cm¹.

HNMR (DMSO-<¼, 500 MHz): 7.76 (dd, J=8.3, 2.2 Hz, 1H), 7.72 (d, J=8.4 Hz, 1H), 7.71 (dd, J=8.9, 5.5 Hz, 2H), 7.39 (t, J=8.8 Hz, 2H), 7.32 (d, J=2.1 Hz, 1H), 4.95 (s, 2H), 4.21 (d, J=2.5 Hz, 2H), 3.25 (t, J=2.5 Hz, 1H ppm).

CNMR (DMSO-i/e, 125 MHz): 170.7, 164.5 (d, J=250.5 Hz), 135.9, 133.4, 132.3 (d, J=9.3 Hz), 131.7, 131.5, 131.5 (d, J=2.9 Hz), 130.4, 128.3, 116.0 (d, J=22.0 Hz), 78.3, 76.1, 54.3, 38.2 ppm.

Elemental analysis [calculated on the basis of the Formula C₁₇H₁₂ClFN₂0₂S (362.81)]

Calculated: C, 56.28; H, 3.33; CI, 9.77; S, 8.84; N, 7.72%.

Measured: C, 56.39; H, 3.34; CI, 9.62; S, 8.99; N, 7.72%.

### Example 20

### 7-Chloro-5-(4-fluoro-phenyl)-3-isopropyl-4,3-dihydro-2,3,4-benzothiadiazepin-2,2-dioxide

The title product is prepared according to the process C/1 starting from 7-chloro-5-(4-fluoro-phenyl)-1,3-dihydro-2,3,4-benzothiadiazepin-2,2-dioxide and isopropyl iodide.

Yield: 82%

Melting point: 147-151° C. (diisopropyl ether)

IR (KBr): 3080, 1601, 1544, 1508, 1323, 1158, 1139, 852 cm^{″1}.

HNMR (CDCI₃, 500 MHz): 7.74 (dd, J=8.8, 5.4 Hz, 2H), 7.53 (dd, J=8.3, 2.2 Hz, 1H), 7.42 (d, J=8.2 Hz, 1H), 7.21 (d, J=2.1 Hz, 1H), 7.16 (t, J=8.5 Hz, 2H), 4.31 (s, 2H), 4.29 (m, 1H), 1.28 (d, J=6.6 Hz, 6H) ppm.

CNMR (CDCI3, 125 MHz): 172.4, 165.0 (d, J=253.9 Hz), 135.8, 134.5, 131.6 (d, J=8.8 Hz), 131.4, 131.4 (d, J=3.4 Hz), 130.3, 129.8, 128.2, 115.9 (d, J=22.0 Hz), 56.4, 50.3, 20.7 ppm.

Elemental analysis [calculated on the basis of the Formula C₁₇H₁₆ClFN₂0₂S (366.84)]

Calculated: C, 55.66; H, 4.40; CI, 9.66; S, 8.74; N, 7.64%.

Measured: C, 55.33; H, 4.41; CI, 9.55; S, 8.60; N, 7.54%.

### Example 21

### 7-Chloro-5-(4-fluoro-phenyl)-3-(3-methyl-but-2-enyl)-1,3-dihydro-2,3,4-benzothiadiazepin-2,2-dioxide

The title product is prepared according to the process C/3 starting from 7-chloro-5-(4-fluoro-phenyl)-1,3-dihydro-2,3,4-benzothiadiazepin-2,2-dioxide and 1-bromo-3-methyl-but-2-ene.

Yield: 93%

Melting point: 168-169° C. (diisopropyl ether)

IR (KBr): 2925, 1598, 1505, 1327, 1155, 1133 cm⁻¹.

HNMR (CDCI3, 500 MHz): 7.66 (dd, J=9.0, 5.4 Hz, 2H), 7.54 (dd, J=8.2, 2.1 Hz, 1H), 7.44 (d, J=8.2 Hz, 1H), 7.22 (d, J=2.2 Hz, 1H), 7.13 (t, J=8.4 Hz, 2H), 5.04 (t, J=1.3 Hz, 1H), 4.33 (s, 2H), 4.16 (d, J=6.9 Hz, 2H), 1.70 (d, J=1.1 Hz, 6H) ppm.

CNMR (CDCI3, 125 MHz): 170.7, 164.9 (d, J=253.9 Hz), 137.7, 135.9, 134.7, 131.6 (d, J=8.8 Hz), 131.4, 131.2 (d, J=3.4 Hz), 130.4, 129.2, 128.5, 117.9, 115.9 (d, J=22.0 Hz), 55.7, 45.88, 25.8, 18.1 ppm.

Elemental analysis [calculated on the basis of the Formula C₁₉H₁₈ClFN₂0₂S (392.88)]

Calculated: C, 58.09; H, 4.62; CI, 9.02; S, 8.16; N, 7.13%.

Measured: C, 58.01; H, 4.68; CI, 8.96; S, 8.15; N, 7.07%.

### Example 22

### 7-Chloro-5-(4-fluoro-phenyl)-3-(3-methyl-butyl)-1,3-dihydro-2,3,4-benzothiadiazepin-2,2-dioxide

The title product is prepared according to the process C/1 starting from 7-chloro-5-(4-fluoro-phenyl)-1,3-dihydro-2,3,4-benzothiadiazepin-2,2-dioxide and 1-bromo-3-methyl-but-2-ene.

Yield: 90%

Melting point: 123-124° C. (diisopropyl ether-hexane)

IR (KBr): 2960, 1603, 1556, 1510, 1326, 1134, 847 cm^{′1}.

HNMR (CDCI3, 500 MHz): 7.72 (dd, 2H, J=8.9, 5.4 Hz), 7.54 (dd, 1H, J=8.2, 2.2 Hz), 7.44 (d, 1H, J=8.3 Hz), 7.23 (d, 1H, J=2.2 Hz), 7.15 (t, 2H, J=8.6 Hz), 4.33 (s, 2H), 3.59 (t, J=7.0 Hz, 2H), 1.39 (m, 1H), 1.34 (m, 2H), 0.74 (d, J=6.3 Hz, 6H) ppm.

CNMR (CDCI3, 125 MHz): 170.9, 165.0 (d, J=254.4 Hz), 135.9, 134.6, 131.6 (d, J=8.8 Hz), 131.4, 131.0 (d, J=2.9 Hz), 130.4, 129.3, 128.2, 116.0 (d, J=22.0 Hz), 55.5, 46.2, 36.6, 25.7, 22.3 ppm.

Elemental analysis [calculated on the basis of the Formula C₁₉H₂₀CIFN₂O₂S

(394.90)]

Calculated: C, 57.79; H, 5.10; CI, 8.98; S, 8.12; N, 7.09%.

Measured: C, 57.56; H, 5.18; CI, 8.93; S, 8.39; N, 7.08%.

### Example 23

### 3-Benzyl-7-chloro-5-(4-fluoro-phenyl)-1,3-dihydro-2̂,4-benzothiadiazepin-2,2-dioxide

The title product is prepared according to the process C/2 starting from 7-chloro-5-(4-fluoro-phenyl)-1,3-dmydro-2,3,4-benzothiadiazepin-2,2-dioxide and benzyl bromide.

Yield: 89%

Melting point: 153° C. (diisopropyl ether)

IR (KBr): 1600, 1506, 1333, 1153, 842 cm^{″1}.

HNMR (CDCI3, 500 MHz): 7.56 (dd, J=8.2, 2.2 Hz, 1H), 7.49 (dd, J=8.9, 5.4 Hz, 2H), 7.20 (m, 3H), 7.05 (t, J=8.6 Hz, 2H), 7.02 (d, J=2.0 Hz, 1H), 7.00 (d, J=2.0 Hz, 2H), 4.73 (s, 2H), 4.40 (s, 2H) ppm.

CNMR (CDCI3, 125 MHz): 171.7, 165.0 (d, J=253.9 Hz), 136.0, 135.5, 134.7, 131.6 (d, J=9.3 Hz), 131.5, 130.8 (d, J=2.9 Hz), 130.4, 129.3, 128.5, 128.3, 128.2, 127.5, 115.8 (d, J=22.0 Hz), 55.7, 51.3 ppm.

Elemental analysis [calculated on the basis of the Formula C₂₁H₁₆ClFN₂0₂S (414.89)]

Calculated: C, 60.80; H, 3.89; CI, 8.55; S, 7.73; N, 6.75%.

Measured: C, 60.73; H, 3.75; CI, 8.42; S, 7.88; N, 6.75%.

### Example 24

### 7-Fluoro-5-(4-fluoro-phenyl)-3-methyl-1,3-dihydro-2,3,4-benzothiadiazepin-2,2-dioxide

The title product is prepared according to the process C/1 starting from 7-fluoro-5-(4-fluoro-phenyl)-1,3-dihydro-2,3,4-benzothiadiazepin-2,2-dioxide and methyl iodide.

Yield: 90%

Melting point: 169-171° C. (ethanol)

IR (KBr): 2992, 2947, 1601, 1555, 1493, 1326, 1263, 1231 cm^{″1}.

HNMR (CDCl₃, 500 MHz): 7.72 (dd, J=8.4, 5.4 Hz, 2H), 7.50 (dd, J=8.4, 5.1 Hz, 1H), 7.28 (td, J=8.3, 2.3 Hz, 1H), 7.14 (t, J=8.3 Hz, 2H), 6.97 (dd, J=8.4, 2.4 Hz, 1H), 4.36 (s, 2H), 3.15 (s, 3H) ppm.

CNMR (CDCI3, 125 MHz): 169.9, 165.0 (d, J=253.9 Hz), 162.0 (d, J=251.0 Hz), 136.1 (d, J=7.8 Hz), 131.7 (d, J=8.8 Hz), 131.1 (d, JM8.8 Hz), 130.9 (d, J=2.9 Hz), 126.5 (d, J=3.4 Hz), 118.6 (d, J=22.0 Hz), 115.9 (d, J=23.4 Hz), 115.9 (d, J=22.0 Hz), 55.1, 35.3 ppm.

Elemental analysis [calculated on the basis of the Formula C₁₅H₁₂F₂N₂0₂S

(322.34)]

Calculated: C, 55.89; H, 3.75; S, 9.95; N, 8.69%.

Measured: C, 56.31; H, 3.73; S, 10.18; N, 8.81%.

### Example 25

### 7-Fluoro-3-ethyl-5-(4-fluoro-phenyl)-1,3-dihydro-2,3,4-benzothiadiazepin-2,2-dioxide

The title product is prepared according to the process C/1 starting from 7-fluoro-5-(4-fluoro-phenyl)-1,3-dihydro-2,3,4-benzothiadiazepin-2,2-dioxide and ethyl iodide.

Yield: 94%

Melting point: 148-151° C. (diisopropyl-ether)

IR (KBr): 2977, 2935, 1602, 1553, 1507, 1330, 1233, 1144 cm⁻¹.

HNMR (DMSO-d₆, 500 MHz): 7.72 (m, 3H), 7.51 (td, J=8.6, 2.7 Hz, 1H), 7.36 (t, J=8.8 Hz, 2H), 7.14 (dd, J=9.0, 2.7 Hz, 1H), 4.80 (s, 2H), 3.44 (q, J=7.0 Hz, 2H), 1.04 (t, J=7.2 Hz, 3H) ppm.

CNMR (DMSO-d₆, 125 MHz): 170.6 (d, J=2.0 Hz), 164.3 (d, J=250.0 Hz), 161.4 (d, J=246.6 Hz), 136.2 (d, J=7.8 Hz), 132.1 (d, J=8.8 Hz), 131.8 (d, J=8.8 Hz), 131.8 (d, J=2.9 Hz), 128.0 (d, J=2.9 Hz), 118.5 (d, J=21.5 Hz), 115.9 (d, J=22.0 Hz), 115.5 (d, J=23.4 Hz), 54.1, 42.32, 13.1 ppm.

Elemental analysis [calculated on the basis of the Formula C₁₆H₁₄F₂N₂0₂S

(336.36)]

Calculated: C, 57.13; H, 4.20; S, 9.53; N, 8.33%.

Measured: C, 56.86; H, 4.25; S, 9.59; N, 8.33%.

### Example 26

### 3-Butyl-7-fluoro-5-(4-fluoro-phenyl)-1,3-dihydro-2,3,4-benzothiadiazepin-2,2-dioxide

The title product is prepared according to the process C/1 starting from 7-fluoro-5-(4-fluoro-phenyl)-1,3-dihydro-2,3,4-benzothiadiazepin-2,2-dioxide and butyl iodide.

Yield: 88%

Melting point: 99-103° C. (ethanol)

IR (KBr): 3072, 2971, 2938, 2909, 2861, 1602, 1549, 1497, 1331, 1233, 1156, 1131, 1080 cm-¹.

HNMR (DMSO-d₆, 500 MHz): 7.72 (m, 3H), 7.51 (td, J=8.7, 2.7 Hz, 1H), 7.36 (t, J=8.9 Hz, 2H), 7.24 (dd, J=9.0, 2.7 Hz, 1H), 4.81 (s, 2H), 3.41 (t, J=7.0 Hz, 2H), 1.40 (m, 2H), 1.05 (m, J=7.0 Hz, 2H), 0.74 (t, 3H) ppm.

CNMR (DMSO-d₆, 125 MHz): 170.8 (d, J=1.5 Hz), 164.3 (d, J=250.5 Hz), 161.3 (d, J=246.6 Hz), 136.3 (d, J=8.3 Hz), 132.0 (d, J=8.8 Hz), 131.7 (d, J=8.8 Hz), 131.7 (d, J=2.9 Hz), 128.2 (d, J=2.9 Hz), 118.5 (d, J=21.5 Hz), 115.9 (d, J=22.0 Hz), 115.3 (d, J=23.4 Hz), 54.1, 46.5, 29.4, 19.1, 13.5 ppm.

Elemental analysis [calculated on the basis of the Formula C₁₈H₁₈F₂N₂0₂S

(364.42)]

Calculated: C, 59.33; H, 4.98; S, 8.80; N, 7.69%.

Measured: C, 59.06; H, 5.04; S, 8.99; N, 7.61%.

### Example 27

### 7-Fluoro-5-(4-fluoro-phenyl)-3-(3-methyl-but-2-enyl)-1,3-dihydro-2,3,4-benzothiadiazepin-2,2-dioxide

The title product is prepared according to the process C/1 starting from 7-fluoro-5-(4-fluoro-phenyl)-1,3-dihydro-2,3,4-benzothiadiazepin-2,2-dioxide and 1-bromo-3-methyl-but-2-ene.

Yield: 91%

Melting point: 154-157° C. (ethanol)

IR (KBr): 2977, 2918, 1599, 1555, 1494, 1322, 1260, 1231, 1156, 1132 cm⁻¹. HNMR (CDCl₃, 500 MHz): 7.67 (dd, J=8.8, 5.5 Hz, 2H), 7.48 (dd, J=8.5, 5.1 Hz, 1H), 7.27 (td, J=8.4, 2.6 Hz, 1H), 7.12 (t, J=8.8 Hz, 2H), 6.95 (dd, J=8.5, 2.7 Hz, 1H), 5.03 (t, J=7.1 Hz, 1H), 4.33 (s, 2H), 4.16 (d, J=7.1 Hz, 2H), 1.69 (s, 6H) ppm.

CNMR (CDCI₃, 125 MHz): 170.8 (d, J=1.5 Hz), 164.9 (d, J=253.9 Hz), 162.0 (d, J=250.5 Hz), 137.6, 136.2 (d, J=7.8 Hz), 131.6 (d, J=8.8 Hz), 131.2 (d, J=3.4 Hz), 131.0 (d, J=7.8 Hz), 126.8 (d, J=3.4 Hz), 118.5 (d, J=21.5 Hz), 117.9, 115.8 (d, J=22.0 Hz), 115.7 (d, J=23.0 Hz), 55.5, 45.8, 25.8, 18.1 ppm.

Elemental analysis [calculated on the basis of the Formula C₁₉H₁₈F₂N₂O₂S

(376.43)]

Calculated: C, 60.63; H, 4.82; S, 8.52; N, 7.44%.

Measured: C, 60.48; H, 4.93; S, 8.42; N, 7.40%.

### Example 28

### 7-Fluoro-5-(4-fluoro-phenyl)-3-(3-methyl-butyl)-1,3-dihydro-2,3,4-benzothiadiazepin-2,2-dioxide

The title product is prepared according to the process C/1 starting from 7-fluoro-5-(4-fluoro-phenyl)-1,3-dihydro-2,3,4-benzothiadiazepin-2,2-dioxide and 1-bromo-3-methyl-but-2-ene.

Yield: 90%

Melting point: 130-133° C. (ethanol)

IR (KBr): 2956, 2923, 2869, 1602, 1555, 1495, 1325, 1263, 1232, 1158, 1133, 1079 cm⁻¹.

HNMR (CDCl₃, 500 MHz): 7.73 (dd, J=8.9, 5.4 Hz, 2H), 7.49 (dd, J=8.5, 5.1 Hz, 1H), 7.28 (td, J=8.3, 2.7 Hz, 1H), 7.14 (t, J=8.7 Hz, 2H), 6.96 (dd, J=8.4, 2.7 Hz, 1H), 4.33 (s, 2H), 3.59 (t, J=7.2 Hz, 2H), 1.39 (m, 1H), 1.34 (q, J=6.8 Hz, 2H), 0.81 (d, J=6.4 Hz, 6H) ppm.

CNMR (CDCI3, 125 MHz): 170.9 (d, J=1.5 Hz), 165.0 (d, J=254.4 Hz), 162.0 (d, J=251.0 Hz), 136.3 (d, J=7.3 Hz), 131.6 (d, J=8.8 Hz), 131.0 (d, J=8.3 Hz), 131.0 (d, J=3.9 Hz), 126.9 (d, J=2.3 Hz), 118.5 (d, J=22.0 Hz), 115.9 (d, J=22.0 Hz), 115.5 (d, J=23.4 Hz), 55.4, 46.1, 36.6, 25.7, 22.3 ppm.

Elemental analysis [calculated on the basis of the Formula C₁₉H₂₀F₂N₂O₂S

(378.44)]

Calculated: C, 60.30; H, 5.33; S, 8.47; N, 7.40%.

Measured: C, 60.10; H, 5.44; S, 8.50; N, 7.37%.

### Example 29

### 7-Fluoro-5-(4-fluoro-phenyl)-3-hexyl-1,3-dihydro-2,3,4-benzothiadiazepin-2,2-dioxide

The title product is prepared according to the process C/2 starting from 7-fluoro-5-(4-fluoro-phenyl)-1,3-dihydro-2,3,4-benzothiadiazepin-2,2-dioxide and hexyl bromide.

Yield: 82%

Melting point: 93-96° C. (IP A)

IR (KBr): 2930, 2858, 1600, 1554, 1495, 1326, 1263, 1231, 1158, 1076 cm⁻¹. HNMR (DMSO-fl?₆, 500 MHz): 7.73 (m, 3H), 7.51 (td, J=8.6, 2.7 Hz, 1H), 7.36 (t, J=8.8 Hz, 2H), 7.11 (dd, J=8.9, 2.7 Hz, 1H), 4.81 (s, 2H), 3.41 (t, J=6.4 Hz, 2H), 1.42 (m, 2H), 1.10 (m, 4H), 0.98 (m, 2H), 0.73 (t, J=7.0 Hz, 3H) ppm.

CNMR (DMSO-d₆, 125 MHz): 171.0 (d, J=1.5 Hz), 164.4 (d, J=250.5 Hz), 161.3 (d, J=247.1 Hz), 136.3 (d, J=7.8 Hz), 132.0 (d, J=8.8 Hz), 131.7 (d, J=8.3 Hz), 131.6 (d, J=2.9 Hz), 128.2 (d, J 2.9 Hz), 118.4 (d, J=22.0 Hz), 115.9 (d, J=22.0 Hz), 115.3 (d, J=23.4 Hz), 54.2, 46.7, 30.7, 27.1, 25.4, 22.1, 13.8 ppm.

Elemental analysis [calculated on the basis of the Formula C₂₀H₂₂F₂N₂0₂S (392.47)]

Calculated: C, 61.21; H, 5.65; S, 8.17; N, 7.14%.

Measured: C, 61.15; H, 5.83; S, 8.13; N, 7.10%.

### Example 30

### 3-Benzyl-7-fluoro-5-(4-fluoro-phenyl)-1,3-dihydro-2,3,4-benzothiadiazepin-2,2-dioxide

The title product is prepared according to the process C/3 starting from 7-fluoro-5-(4-fluoro-phenyl)-1,3-dihydro-2,3,4-benzothiadiazepin-2,2-dioxide and benzyl bromide.

Yield: 90%

Melting point: 184-187° C. (ethanol)

IR (KBr): 3430, 2995, 2949, 2926, 1598, 1547, 1494, 1320, 1232, 1153, 1129, 1074 cm⁻¹.

HNMR (DMSO-d₆, 500 MHz): 7.77 (dd, J=8.6, 5.4 Hz, 1H), 7.55 (td, J=8.7, 2.7 Hz, IK), 7.53 (dd, J=9.0, 5.5 Hz, 2H), 7.30 (t, J=8.8 Hz, 2H), 7.24 (t, J=1.8 Hz, 2H), 7.21 (t, J=1.5 Hz, IK), 7.00 (d, J=2.5 Hz, 2H), 6.99 (dd, J=9.0, 2.7 Hz, 1H), 4.92 (s, 2H), 4.64 (s, 2H) ppm.

CNMR (DMSO-d₆, 125 MHz): 171.1 (d, J=1.5 Hz), 164.3 (d, J=250.0 Hz), 161.4 (d, J=247.6 Hz), 136.3 (d, J=7.8 Hz), 136.2, 132.0 (d, J=9.3 Hz), 131.9 (d, J=8.8 Hz), 131.4 (d, J=2.9 Hz), 128.4, 128.2 (d, J=2.0 Hz), 128.1, 127.4, 118.6 (d, J=22.0 Hz), 115.9 (d, J=22.0 Hz), 115.2 (d, J=23.4 Hz), 54.1, 50.8 ppm.

Elemental analysis [calculated on the basis of the Formula C₂₁H₁₆F₂N₂0₂S

(398.44)]

Calculated: C, 63.31; H, 4.05; S, 8.05; N, 7.03%.

Measured: C, 63.03; H, 4.03; S, 7.91; N, 6.98%.

### Example 31

### 7-Fluoro-5-(4-fluoro-phenyl)-3-(3-phenyl-propyl)-1,3-dihydro-2,3,4-benzothiadiazepin-2,2-dioxide

The title product is prepared according to the process C/1 starting from 7-fluoro-5-(4-fluoro-phenyl)-1,3-dihydro-2,3,4-benzothiadiazepin-2,2-dioxide and 3-phenyl-propyl bromide.

Yield: 80%

Melting point 163-167° C. (ethanol)

IR (KBr): 2992, 2939, 2867, 1601, 1557, 1495, 1451, 1424, 1329, 1262, 1230, 1158, 1059 cm-¹.

HNMR (DMSO-d₆, 500 MHz): 7.74 (m, 3H), 7.53 (td, J=8.7, 2.7 Hz, 1H), 7.37 (t, J=8.8 Hz, 2H), 7.22 (t, J=7.4 Hz, 2H), 7.20 (dd, J=8.9, 2.7 Hz, 1H), 7.14 (t, J=7.3 Hz, 1H), 7.02 (d, J=7.1 Hz, 2H), 4.84 (s, 2H), 3.43 (t, J=6.8 Hz, 2H), 2.31 (t, J=7.7 Hz, 2H), 1.72 (qn, J=7.8 Hz, 2H) ppm.

CNMR DMSO-d₆, 125 MHz): 171.2 (d, J=1.5 Hz), 164.4 (d, J=250.0 Hz), 161.4 (d, J=247.1 Hz), 141.3, 136.3 (d, J=7.8 Hz), 132.1 (d, J=9.3 Hz), 131.8 (d, J=8.8 Hz), 131.6 (d, J=2.9 Hz), 128.4, 128.4, 128.2 (d, J=2.9 Hz), 125.9, 118.5 (d, J=21.5 Hz), 115.9 (d, J=22.0 Hz), 115.4 (d, J=23.9 Hz), 54.2, 46.2, 31.9, 29.4 ppm.

Elemental analysis [calculated on the basis of the Formula C₂₃H₂₀F₂N₂O₂S

(426.49)]

Calculated: C, 64.77; H, 4.73; S, 7.52; N, 6.57%.

Measured: C, 64.70; H, 4.89; S, 7.58; N, 6.56%.

### Example 32

### 5-(3-Fluoro-phenyl)-7-chloro-3-methyl-1,3-dihydro-2,3,4-benzothiadiazepin-2,2-dioxide

The title product is prepared according to the process C/1 starting from 7-chloro-5-(3-fluoro-phenyl)-1,3-dihydro-2,3,4-benzothiadiazepin-2,2-dioxide and methyl iodide.

Yield: 85%

Melting point 172-173° C. (ethanol)

IR (KBr): 2992, 2939, 2867, 1601, 1557, 1495, 1451, 1424, 1329, 1262, 1230, 1158, 1059 cm⁻¹.

HNMR (CDCl₃, 500 MHz): 7.56 (dd, J=8.2, 2.2 Hz, 1H), 7.49 (m, 1H), 7.45 (d, J=8.2 Hz, 1H), 7.41 (m, 2H), 7.25 (m, 2H), 4.36 (s, 2H), 3.16 (s, 3H) ppm.

CNMR (CDCI3, 125 MHz): 169.7 (d, J=2.9 Hz), 162.8 (d, J=248.0 Hz), 136.8 (d, J=7.3 Hz), 135.4, 134.9, 131.7, 130.5, 130.3 (d, J=7.8 Hz), 128.9, 128.6, 125.5 (d, J=2.9 Hz), 118.9 (d, J=21.5 Hz), 116.0 (d, J=23.4 Hz), 55.3, 35.4.

Elemental analysis [calculated on the basis of the Formula C₁₅H₁₂ClFN₂0₂S (338.79)]

Calculated: C, 53.18; H, 3.57; CI, 10.46; S, 9.46; N, 8.27%.

Measured: C, 53.00; H, 3.60; CI, 10.37; S, 9.53; N, 8.17%.

### Example 33

### 8-Cyano-5-(4-fluoro-phenyl)-3-methyl-1,3-dihydro-2,3,4-benzothiadiazepin-2,2-dioxide

The title product is prepared according to the process C/1 starting from 7-chloro-5-(3-fluoro-phenyl)-1,3-dihydro-2,3,4-benzothiadiazepin-2,2-dioxide and methyl iodide.

Yield: 80%

Melting point 284-285° C. (methanol)

HNMR (CDCl₃, 500 MHz): 7.81 (d, J=1.0 Hz, IH), 7.78 (dd, J=8.1, 1.6 Hz, 1H), 7.69 (dd, J=8.9, 5.3 Hz, IH), 7.15 (t, J=8.6 Hz, 2H), 4.41 (s, 2H), 3.15 (s, 3H) ppm.

CNMR (CDCI3, 125 MHz): 169.7, 165.2 (d, J=254.9 Hz), 138.3, 132.4, 132.2, 131.8, 131.6 (d, J=9.3 Hz), 130.4 (d, J=2.9 Hz), 129.6, 117.1, 116.1 (d, J=22.0 Hz), 115.4, 55.2, 35.3 ppm.

Elemental analysis [calculated on the basis of the Formula C₁₆H₁₂FN₃0₂S (329.36)]

Calculated: C, 58.35; H, 3.67; S, 9.74; N, 12.76%.

Measured: C, 57.96; H, 3.69; S, 9.87; N, 12.62%.

### Example 34

### 8-Fluoro-5-(4-fluoro-phenyl)-3-methyl-1,3-dihydro-2,3,4-benzothiadiazepin-2,2-dioxide

The title product is prepared according to the process C/1 starting from 8-fluoro-5-(4-fluoro-phenyl)-1,3-dihydro-2,3,4-benzothiadiazepin-2,2-dioxide and methyl iodide.

Yield: 92%

Melting point 205-206° C. (methanol)

HNMR (CDCI3, 500 MHz): 7.70 (dd, J=8.4, 5.4 Hz, 2H), 7.25 (m, 2H), 7.18 (dt, J=8.3, 2.4 Hz, IH), 7.12 (t, J=8.4 Hz, 2H), 4.35 (s, 2H), 3.14 (d, J=0.6 Hz, 3H) ppm.

CNMR (CDCl₃, 125 MHz): 170.3, 165.0 (d, J=253.4 Hz), 163.9 (d, J=253.9 Hz), 132.9 (d, J=8.8 Hz), 131.7 (d, J=8.8 Hz), 131.3 (d, J=3.4 Hz), 131.1 (d, J=9.3 Hz), 130.4 (d, J=3.4 Hz), 116.5 (d, J=22.9 Hz), 115.9 (d, J=22.0 Hz), 115.8 (d, J=22.0 Hz), 55.5 (d, J=2.0 Hz), 35.3 ppm.

Elemental analysis [calculated on the basis of the Formula C₁₅H₁₂F₂N₂0₂S (322.34)]

Calculated: C, 55.89; H, 3.75; S, 9.95; N, 8.69%.

Measured: C, 55.40; H, 3.67; S, 9.95; N, 8.57%.

### Example 35

### 5-(4-Fluoro-phenyl)-8-chloro-3-methyl-1,3-dihydro-2,3,4-benzothiadiazepin-2,2-dioxide

The title product is prepared according to the process C/1 starting from 8-chloro-5-(4-fluoro-phenyl)-1,3-dmydro-2,3,4-benzothiadiazepin-2,2-dioxide and methyl iodide.

Yield: 90%

Melting point 250-251° C. (methanol)

HNMR (CDCl₃, 500 MHz): 7.70 (dd, J=8.9, 5.4 Hz, 2H), 7.52 (d, J=2.0 Hz, 1H), 7.46 (dd, J=8.3, 2.0 Hz, 1H), 7.20 (d, J=8.3 Hz, 1H), 7.13 (t, J=8.5 Hz, 2H) ppm. CNMR (CDCI3, 125 MHz): 170.3, 165.0 (d, J=253.9 Hz), 137.7, 132.5, 132.1, 131.7 (d, J=9.3 Hz), 131.1 (d, J=3.4 Hz), 130.1, 129.3, 128.9, 115.9 (d, J=21.7 Hz), 55.4, 35.3 ppm.

Elemental analysis [calculated on the basis of the Formula C₁₅H₁₂ClFN₂0₂S (338.79)]

Calculated: C, 53.18; H, 3.57; CI, 10.46; S, 9.46; N, 8.27%.

Measured: C, 52.79; H, 3.62; CI, 10.90; S, 9.90; N, 8.16%.

### Example 36

### 5-(4-Fluoro-phenyl)-3-methyl-8-trifluoromethyl-1,3-dihydro-2,3,4-benzothiadiazepin-2,2-dioxide

The title product is prepared according to the process C/1 starting from 5-(4-fluoro-phenyl)-8-(trifluoromethyl)-1,3-dihydro-2,3,4-berizothiadiazepin-2,2-dioxide and methyl iodide.

Yield: 88%

Melting point 160-162° C. (methanol)

HNMR (CDCl₃, 500 MHz): 7.78 (s, 1H), 7.75 (d, J=8.7 Hz, IH), 7.71 (dd, J=8.7, 5.4, 2H), 7.41 (d, J=7.9 Hz, IH), 7.14 (t, J=8.5 Hz, 2H), 4.44 (s, 2H), 3.16 (s, 3H) ppm.

CNMR (CDCl₃, 125 MHz): 170.0, 165.1 (d, J=254.4 Hz), 137.5, 133.4 (q, J=33.2 Hz), 131.7 (d, J=9.3 Hz), 131.4, 130.8 (d, J=3.4 Hz), 129.4, 126.0 (d, J=3.7 Hz), 125.6 (q, J=3.6 Hz), 123.2 (q, J=272.9 Hz), 116.0 (d, J=22.0 Hz), 55.6, 35.3 ppm.

Elemental analysis [calculated on the basis of the Formula C₁₆H₁₂F₄N₂0₂S

(372.34)]

Calculated: C, 51.61; H, 3.25; S, 8.61; N, 7.52%.

Measured: C, 51.52; H, 3.32; S, 8.54; N, 7.46%.

### Example 37

### 7,8-Dichloro-5-(4-fluoro-phenyl)-3-methyl-1,3-dihydro-2,3,4-benzothiadiazepin-2,2-dioxide

The title product is prepared according to the process C/1 starting from 8-dichloro-5-(4-fluoro-phenyl)-1,3-dihydro-2,3,4-benzothiadiazepin-2,2-dioxide and methyl iodide.

Yield: 92%

Melting point 236-239° C. (EPA-DMF)

IR (KBr): 2970, 2921, 1601, 1546, 1509, 1475, 1328, 1229, 1159, 1136, 1071 cm⁻¹. HNMR (DMSO-d₆, 500 MHz): 8.03 (s, 0.1H), 7.72 (dd, J=8.9, 5.5 Hz, 2H), 7.54 (s, 1H), 7.36 (t, J=8.9 Hz, 2H), 4.90 (s, 2H), 3.01 (s, 3H) ppm.

CNMR (DMSO-d₆, 125 MHz): 169.1, 164.4 (d, J=250.5 Hz), 134.4, 134.1, 132.3 (d, J=8.8 Hz), 132.0, 131.4, 131.3 (d, J=2.9 Hz), 131.2, 130.49, 115.9 (d, J=22.0 Hz), 53.5, 34.9 ppm.

Elemental analysis [calculated on the basis of the Formula C₁₆H₁₂F₄N₂0₂S (372.34)]

Calculated: C, 48.27; H, 2.97; CI, 19.00; S, 8.59; N, 7.51%.

Measured: C, 48.31; H, 2.99; CI, 18.96; S, 8.60; N, 7.53%.

### Example 38

### 9-(4-fluorophenyl)-7-methyl-5H,7H-[1,2,5]thiadiazolo[3,4-h][2,3,4]benzothiadiazepin-6,6-dioxide

The title product is prepared according to the process C/1 starting from 9-(4-fluorophenyl)-5H,7H-[1,2,5]thiadiazolo[3,4-/z][2,3,4]benzothiadiazepin-6,6-dioxide and methyl iodide.

Yield: 95%

Melting point 272-275° C. (ethanol-DMF)

IR (KBr): 3137, 1597, 1569, 1508, 1326 cm-¹.

HNMR (DMSO-d₆, 500 MHz): 10.34 (s, 1H), 8.38 (s, 1H), 8.02 (s, 1H), 7.80 (dd, J=8.7, 5.5 Hz, 2H), 7.36 (t, J=8.9 Hz, 2H), 4.96 (s, 2H) ppm.

CNMR (DMSO-d₆, 125 MHz): 172.0, 164.3 (d, J=250.0 Hz), 154.4, 153.0, 136.3, 133.1, 132.4 (d, J=2.9 Hz), 132.2 (d, J=8.7 Hz), 122.6, 121.5, 115.8 (d, J=22.0 Hz), 55.1 ppm.

Elemental analysis [calculated on the basis of the Formula C₁₅H₁₁FN₄0₂S₂

(362.41)]

Calculated: C, 49.71; H, 3.06; N, 15.46; S, 17.70%.

Measured: C, 50.04; H, 3.06; N, 15.82; S, 17.50%.

### Example 39

### 7,8-Dichloro-3-ethyl-5-(4-fluoro-phenyl)-1,3-dihydro-2,3,4-benzothiadiazepin-2,2-dioxide

The title product is prepared according to the process C/3 starting from 7,8-dichloro-5-(4-fluoro-phenyl)-1,3-dihydro-2,3,4-benzothiadiazepin-2,2-dioxide and ethyl iodide.

Yield: 76%

Melting point 218-221° C. (acetonitrile)

IR (KBr): 2986, 1601, 1326, 1138 cm^{″1}.

HNMR (DMSO-d₆, 500 MHz): 8.03 (s, 1H), 7.74 (dd, J=9.0, 5.5 Hz, 2H), 7.55 (s, 1H), 7.36 (t, J=8.8 Hz, 2H), 4.86 (s, 2H), 3.46 (q, 2H), 1.05 (t, 3H) ppm.

CNMR (DMSO-d₆, 125 MHz): 170.07, 164.43 (d, J=250.5 Hz), 134.60, 134.10, 132.22 (d, J=9.3 Hz), 132.21, 131.57 (d, J=2.9 Hz), 131.44, 131.17, 130.29, 115.92 (d, J=22.0 Hz), 53.81, 42.46, 13.08 ppm.

Elemental analysis [calculated on the basis of the Formula C₁₆H₁₃Cl₂FN₂0₂S (387.26)]

Calculated: C, 49.62; H, 3.38; S, 8.28; CI, 18.31; N, 7.23%.

Measured: C, 49.60; H, 3.45; S, 8.19; CI, 18.03; N, 7.26%.

C(1)-alkylating reaction (Process “D”)

15 mmol of 1,3-dihydro-2,3,4-benzothiadiazepine-2,2-dioxide, alkylated (aralkylated) in position 3, appropriately substituted on the aromatic carbocycle and containing an appropriately substituted phenyl group in position 5 is added to 100 ml tetrahydrofurane. To the solution is added 7 ml (17.5 mmol) 2.5 M BuLi in hexane under stirring in an acetone-dry ice bath at −78° C.

After stirring for one hour at this temperature, 16.5 mmol alkyl iodide is added to the red solution in 8 ml tetrahydrofurane. After warming to room temperature, the reaction mixture is added to 150 ml ice and stirred half an hour. After extracting the aqueous mixture with 180 ml ethyl acetate, the organic layer is extracted with brine, dried over MgS0₄ and evaporated. The product is crystallized. The solvent used by crystallization is indicated at the melting points of each compound.

### Example 40

### 7-Chloro-5-(4-fluoro-phenyl)-1,3-dimethyl-1,3-dihydro-2,3,4-benzothiadiazepin-2,2-dioxide

The title product is prepared according to the process D starting from 7-chloro-5-(4-fluoro-phenyl)-3-methyl-1,3-dihydro-2,3,4-benzothiadiazepin-2,2-dioxide and methyl iodide.

Yield: 87%

Melting point: 139-143° C. (2-propanol)

IR (KBr): 2937, 1602, 1550, 1508, 1317, 1236, 1143, 1041 cm^{′1}.

HNMR (CDCl₃, 500 MHz): 7.73 (dd, J=9.0, 5.4 Hz, 2H), 7.58 (dd, J=8.4, 2.2 Hz, IH), 7.45 (d, J=8.4 Hz, 1H), 7.20 (d, J=2.2 Hz, IH), 7.14 (t, J=9.0 Hz, 2H), 4.36 (q, J=7.1 Hz, 1H), 3.16 (s, 3H), 1.88 (d, J=7.1 Hz, 3H) ppm.

CNMR (CDCl₃, 125 MHz): 169.6, 165.0 (d, J=253.9 Hz), 136.1, 134.4, 133.4, 131.7 (d, J=8.8 Hz), 131.3, 130.9 (d, J=3.4 Hz), 128.4, 127.1, 115.9 (d, J=22.0 Hz), 57.4, 35.9, 10.4 ppm.

Elemental analysis [calculated on the basis of the Formula C₁₆H₁₄ClFN₂0₂S (352.82)]

Calculated: C, 54.47; H, 4.00; CI, 10.05; S, 9.09; N, 7.94%.

Measured: C, 54.56; H, 4.09; CI, 9.90; S, 9.00; N, 7.93%.

### Example 41

### 7-Chloro-1-ethyl-5-(4-fluoro-phenyl)-3-methyl-1,3-dihydro-2,3,4-benzothiadiazepin-2,2-dioxide

The title product is prepared according to the process D starting from 7-chloro-5-(4-fluoro-phenyl)-3-methyl-1,3-dihydro-2,3,4-benzothiadiazepin-2,2-dioxide and ethyl iodide.

Yield: 92%

Melting point: 175-177° C. (ΓPA)

IR (KBr): 2970, 2935, 1603, 1509, 1316, 1146, 841 cm⁻¹.

HNMR (CDCI3, 500 MHz): 7.80-7.70 (m, 2H), 7.6-7.4 (m, 2H), 7.21 (d, J=2.2 Hz, 1H), 7.14 (t, J=8.4 Hz, 2H), 4.16 (q, J=4.5 Hz, 1H), 3.14 (bs, 3H), 2.52-2.35 (m, 1H), 2.34-2.10 (m, 1H). 1.06 (t, J=7.3 Hz, 3H) ppm.

CNMR (CDCI3, 125 MHz): 169.4 (d, J=102.5 Hz), 162.3, 136.0, 134.1, 132.4, 131.4 (d, J=9.1 Hz), 131.0 (d, J=12.5 Hz), 128.5, 128.2, 115.8 (d, J=21.9 Hz), 77.0 (t, J=32.0 Hz), 65.0, 35.6, 11.8 ppm.

Elemental analysis [calculated on the basis of the Formula C₁₇H₁₆ClFN₂0₂S (366.84)]

Calculated: C, 55.66; H, 4.40; CI, 9.66; S, 8.74; N, 7.64%.

Measured: C, 55.40; H, 4.39; CI, 9.50; S, 8.66; N, 7.63%.

### Example 42

### Preparation of the starting material of the Formula (IV) designated Compound 9 [2-(chloromethyl)-5-fluorophenyl](4-fluorophenyl)methanone (9)

A solution of 4-fluoro phenylmagnesium bromide (69 mmol, 7) in 60 cm³ diethylether was added to a cooled solution of 2-(chloromethyl)-5-fluorobenzoyl chloride (61 mmol, 8) in 120 cm³ toluene under −5° C. The mixture was stirred for 3 hours, while the temperature was raised to 25° C. by itself. A 10% aqueous HCl solution was added to quench the reaction mixture and the stirring was continued for half an hour. The phases were separated and the aqueous phase was extracted with 30 cm³ diethylether. The combined organic phases were washed with saturated NaCl solution and dried on MgSO₄. After filtration the solvents were evaporated in vacuo to give a brownish oil.

Yield: 14.6 g (90%)

IR (KBr): 3074, 1668, 1598 cm-1.

HNMR (CHCl3, 200 MHz): 7.87 (m, 2H), 7.53 (m, 1H), 7.17 (m, 2H), 7.12 (m, 2H), 4.69 (s, 2H) ppm.

1. Compounds of the general Formula (I), wherein
‘a’ represents single or double bond,
R¹ and R² represent, independently, hydrogen, halogen, cyano group, C₁₋₆ alkyl group, C₁₋₆ alkoxy group trifluoromethyl group or other C₁₋₆ alkyl group substituted by one or more halogen atom
or if
‘a’ represents single bond,
R¹ and R² together form a group of the formula ═N—S—N═ attached to the atoms of the bond ‘a’ according to the partial Formula (A)
R³ and R⁴ represents, independently, hydrogen C₁₋₆ alkyl group, C₁₋₆ alkenyl group, C₃₋₆ cycloalkyl group, C₁₋₆ aryl group or C₁₋₆ aralkyl group,
R⁵ and R⁶ represents, independently, hydrogen, halogen, cyano group, C₁₋₆ alkyl group, C₁₋₆ alkoxy group, trifluoromethyl group or other C₁₋₆ alkyl group substituted by one or more halogen atom
enantiomers and pharmaceutically suitable acid addition salts thereof.

2. A compound selected from
8-cyano-5-(4-fluoro-phenyl)-1,3-dihydro-2,3,4-benzothiadiazepine-2,2-dioxide;
7-chloro-5-(3-fluoro-phenyl)-1,3-dihydro-2,3,4-benzothiadiazepine-2,2-dioxide,
9-(4-methyl-phenyl)-5H,7H-[1,2,5]thiadiazolo[3,4-h]-2,3,4-benzothiadiazepine-6,6-dioxide;
9-(4-methoxy-phenyl)-5H,7H-[1,2,5]thiadiazolo[3,4-h][2,3,4]benzothiadiazepine-6,6-dioxide;
9-(3,4-dimethoxy-phenyl)-5H,7H-[1,2,5]thiadiazolo[3,4-h][2,3,4]benzothiadiazepine-6,6-dioxide;
9-(4-fluoro-phenyl)-5H,7H-[1,2,5]thiadiazolo[3,4-h][2,3,4]benzothiadiazepine-6,6-dioxide;
7-chloro-5-(4-fluoro-phenyl)-1,3-dihydro-2,3,4-benzothiadiazepien-2,2-dioxide;
7-fluoro-5-(4-fluoro-phenyl)-1,3-dihydro-2,3,4-benzothiadiazepine-2,2-dioxide;
7,8-dichloro-5-(4-fluoro-phenyl)-1,3-dihydro-2,3,4-benzothiadiazepine-2,2-dioxide;
7-chloro-5-(4-fluoro-phenyl)-3-methyl-1,3-dihydro-2,3,4-benzothiadiazepine-2,2-dioxide;
7-chloro-3-ethyl-5-(4-fluoro-phenyl)-1,3-dihydro-2,3,4-benzothiadiazepine-2,2-dioxide;
7-chloro-5-(4-fluoro-phenyl)-3-propyl-1,3-dihydro-2,3,4-benzothiadiazepine-2,2-dioxide;
7-chloro-5-(4-fluoro-phenyl)-3-(prop-2-inyl)-1,3-dihydro-2,3,4-benzothiadiazepine-2,2-dioxide;
7-chloro-5-(4-fluoro-phenyl)-3-isopropyl-1,3-dihydro-2,3,4-benzothiadiazepine-2,2-dioxide;
7-chloro-5-(4-fluoro-phenyl)-3-(3-methyl-but-2-enyl)-1,3-dihydro-2,3,4-benzothiadiazepine-2,2-dioxide;
7-chloro-5-(4-fluoro-phenyl)-3-(3-methyl-butyl)-1,3-dihydro-2,3,4-benzothiadiazepine-2,2-dioxide;
3-benzyl-7-chloro-5-(4-fluoro-phenyl)-1,3-dihydro-2,3,4-benzothiadiazepine-2,2-dioxide;
7-fluoro-5-(4-fluoro-phenyl)-3-methyl-1,3-dihydro-2,3,4-benzothiadiazepine-2,2-dioxide;
7-fluoro-3-ethyl-5-(4-fluoro-phenyl)-1,3-dihydro-2,3,4-benzothiadiazepine-2,2-dioxide;
3-butyl-7-fluoro-5-(4-fluoro-phenyl)-1,3-dihydro-2,3,4-benzothiadiazepine-2,2-dioxide;
7-fluoro-5-(4-fluoro-phenyl)-3-(3-methyl-but-2-enyl)-1,3-dihydro-2,3,4-benzothiadiazepine-2,2-dioxide;
7-fluoro-5-(4-fluoro-phenyl)-3-(3-methyl-butyl)-1,3-dihydro-2,3,4-benzothiadiazepine-2,2-dioxide;
7-fluoro-5-(4-fluoro-phenyl)-3-hexyl-1,3-dihydro-2,3,4-benzothiadiazepine-2,2-dioxide;
3-benzyl-7-fluoro-5-(4-fluoro-phenyl)-1,3-dihydro-2,3,4-benzothiadiazepine-2,2-dioxide;
7-fluoro-5-(4-fluoro-phenyl)-3-(3-phenyl-propyl)-1,3-dihydro-2,3,4-benzothiadiazepine-2,2-dioxide;
5-(3-fluoro-phenyl)-7-chloro-3-methyl-1-dihydro-2,3,4-benzothiadiazepine-2,2-dioxide;
8-cyano-5-(4-fluoro-phenyl)-3-methyl-1,3-dihydro-2,3,4-benzothiadiazepine-2,2-dioxide;
8-fluoro-5-(4-fluoro-phenyl)-3-methyl-1,3-dihydro-2,3,4-benzothiadiazepine-2,2-dioxide;
5-(4-fluoro-phenyl)-8-chloro-3-methyl-1,3-dihydro-2,3,4-benzothiadiazepine-2,2-dioxide;
5-(4-fluoro-phenyl)-3-methyl-8-trifluormethyl-1,3-dihydro-2,3,4-benzothiadiazepine-2,2-dioxide;
7,8-dichloro-5-(4-fluoro-phenyl)-3-methyl-1,3-dihydro-2,3,4-benzothiadiazepine-2,2-dioxide;
9-(4-fluoro-phenyl)-7-methyl-5H,7H-[1,2,5]thiadiazolo[3,4-h][2,3,4]benzothiadiazepine-6,6-dioxide;
7,8-dichloro-3-ethyl-5-(4-fluoro-phenyl)-1,3-dihydro-2,3,4-benzothiadiazepine-2,2-dioxide;
7-chloro-5-(4-fluoro-phenyl)-1,3-dimethyl-1,3-dihydro-2,3,4-benzothiadiazepine-2,2-dioxide;
7-chloro-1-ethyl-5-(4-fluoro-phenyl)-3-methyl-1,3-dihydro-2,3,4-benzothiadiazepine-2,2-dioxide
and/or enantiomers and/or pharmaceutically acceptable acid addition salts thereof.

3. Process for the praparation of the compounds of the general Formula (I), wherein R³ and R⁴ are hydrogen and R¹, R², R⁵ and R⁶ are defined in claim 1 and pharmaceutically suitable acid addition salts thereof, by reacting the compound of the Formula (II), wherein R¹, R², R⁵ and R⁶ are defined in claim 1 and M represents monovalent or multivalent metals, with tionyl chloride and by subsequent reaction of the obtained compound of the general Formula (III), wherein R¹, R², R⁵ and R⁶ are defined in claim 1, with hydrazine.

4. Process for the preparation of the compounds of the general Formula (I), wherein R⁴ is hydrogen and R¹, R², R⁵, R⁶ and R³ are defined in claim 1 provided that R³ is different from hydrogen, and pharmaceutically suitable acid addition salts thereof, by reacting the 2,3,4-benzothiadiazepine-dioxide derivatives of the general Formula (I) wherein R³ and R⁴ are hydrogen, R¹, R², R⁵ and R⁶ are defined in claim 1, with agents suitable for introducing an alkyl group, alkenyl group or aralkyl group, in the presence of a base.

5. Process for the preparation of the compounds of the general Formula (I), wherein R⁴ is hydrogen and R¹, R², R⁵, R⁶ and R³ are defined in claim 1 provided that R³ is different from hydrogen, and pharmaceutically suitable acid addition salts thereof, by reacting compounds of the general formula (III), wherein R¹, R², R⁵ and R⁶ are defined in claim 1, with a substituted hydrazine of general formula (VI), wherein R³ is defined in claim 1.

6. Process for the preparation of the compounds of the general Formula (I), wherein R³ and R⁴ are defined in claim 1 provided that R³ and R⁴ are identical and different from hydrogen, and R¹, R², R⁵ and R⁶ are defined in claim 1, and pharmaceutically suitable acid addition salts thereof, by reacting the 2,3,4-benzothiadiazepine-dioxide derivatives of the general Formula (I), wherein R³ and R⁴ are hydrogen, R¹, R², R⁵ and R⁶ are defined in claim 1, with agents suitable for introducing an alkyl group, alkenyl group or aralkyl group, in the presence of a base.

7. Process for the preparation of the compounds of the general Formula (I), wherein R³ and R⁴ are defined in claim 1 provided that R³ and R⁴ are different from hydrogen and R¹, R², R⁵ and R⁶ are defined in claim 1 and pharmaceutically suitable acid addition salts thereof, by reacting the 2,3,4-benzothiadiazepine-dioxide derivatives of the general Formula (I) wherein R⁴ is hydrogen, R¹, R², R⁵, R⁶ and R³ are defined in claim 1 provided that R³ is different from hydrogen, with agents suitable for introducing an alkyl group, alkenyl group or aralkyl group, in the presence of a base.

8. The compounds of general Formula (III), wherein R¹, R², R⁵ and R⁶ are defined in claim 1.

9. Process for the preparation of the compounds of general Formula (III), wherein R¹, R², R⁵ and R⁶ are defined in claim 1 which comprises reacting the compound of general Formula (II), wherein R¹, R², R⁵ and R⁶ are defined in claim 1 with thionyl chloride.

10. The compounds of general Formula (II), wherein R¹, R², R⁵ and R⁶ are defined in claim 1, M represents metal atom, preferably alkali metal atom, more preferably sodium atom.

11. Compounds as defined in claim 10 such as
sodium [4-chloro-2-(4-fluoro-benzoyl)-phenyl]-methanesulfonate;
sodium [6-[(3,4-dimethoxy-phenyl)-carbonyl]-2,1,3-benzothiadiazole-5-yl]-methanesulfonate;
sodium [6-[(4-fluoro-phenyl)-carbonyl]-2,1,3-benzothiadiazole-5-yl]-methanesulfonate;
sodium-[6-[(4-methoxy-phenyl)-carbonyl]-2,1,3-benzothiadiazole-5-yl]-methanesulfonate.

12. Process for the preparation of the derivatives of general Formula (II), wherein R¹, R², R⁵ and R⁶ are defined in claim 1, which comprises reacting the compound of general Formula (IV), wherein R¹, R², R⁵ and R⁶ are defined in claim 1 with ammonium sulfite or alkali sulfite in the present of water or water-immiscible solvent, preferably in acetone, dioxane or in an aliphatic alcohol comprising 1 to 4 carbon atoms or in the mixture thereof.

13. Compounds of the general Formula (I) as defined in claim 1 for the use as a medicament.

14. Pharmaceutical compositions comprising as active ingredient one or more compounds of general Formula (I) as defined in claims 1 and/or enantiomers and/or pharmaceutically suitable acid addition salts thereof in a therapeutically effective dose together with one or more pharmaceutically applicable diluent, excipient and/or inert carrier.

15. Use of the pharmaceutical composition as defined in claim 16 for the treatment and/or prevention of diseases of the central nervous system.

16. Compounds of the general Formula (I) as defined in claim 1 for the use as a medicament in the treatment and/or prevention of diseases of the central nervous system.

17. Method of treatment which comprises administering a patient (in need of such treatment), suffering from central nervous system disorders, a therapeutically effective dose of one or more compounds of general Formula (I) as defined in claim 1 and/or enantiomers and/or pharmaceutically suitable acid additional salts thereof.
